(19) United States
(12) Patent Application Publication
(10) Pub. No.: US 2006/0083772 A1
(43) Pub. Date: Apr. 20, 2006

(54) Coating compositions for bioactive agents

(76) Inventors: David M. DeWitt, Minneapolis, MN (US); Michael J. Finley, Saint Louis Park, MN (US); Laurie R. Lawin, New Brighton, MN (US)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(21) Appl. No.: 11/244,823

(22) Filed: Oct. 6, 2005

Related U.S. Application Data
(63) Continuation-in-part of application No. 11/099,939, filed on Apr. 6, 2005.
(63) Continuation-in-part of application No. 11/099,911, filed on Apr. 6, 2005.
(63) Continuation-in-part of application No. 11/099,935, filed on Apr. 6, 2005.
(63) Continuation-in-part of application No. 11/099,910, filed on Apr. 6, 2005.
(63) Continuation-in-part of application No. 11/099,997, filed on Apr. 6, 2005.
(63) Continuation-in-part of application No. 11/099,796, filed on Apr. 6, 2005.
(60) Provisional application No. 60/559,821, filed on Apr. 6, 2004.

(51) Int. Cl. A61F 2/00

(52) U.S. Cl. 424/426

(57) ABSTRACT

A coating composition and related method for use in applying a bioactive agent to a surface in a manner that will permit the bioactive agent to be released from the coating in vivo. The composition is particularly well suited for coating the surface of implantable medical device, such as a stent or catheter, in order to permit the device to release bioactive agent to the surrounding tissue over time. The composition includes a plurality of compatible polymers having different properties that can permit them to be combined together to provide an optimal combination of such properties as durability, biocompatibility, and release kinetics.

### RELATED APPLICATIONS

This application is a continuation-in-part of U.S. patent application Ser. Nos. 11/099,939, 11/099,911, 11/099,935, 11/099,910, 11/099,997 and 11/099,796, each titled Coating Compositions for Bioactive Agents and each filed Apr. 6, 2005, each of which claims the benefit of U.S. Provisional Application Ser. No. 60/559,821, titled Coating Compositions for Bioactive Agents, filed Apr. 6, 2004, the contents of each of which are hereby incorporated by reference.

### TECHNICAL FIELD

In one aspect, the present invention relates to a method of treating implantable medical devices with coating compositions to provide for the controlled release of bioactive (e.g., pharmaceutical) agents from the surface of the devices under physiological conditions. In another aspect, the invention relates to the coating compositions, per se. In yet another aspect, the invention relates to devices or surfaces coated with such compositions. In yet another aspect, the present invention relates to the local administration of bioactive agents for the prevention and treatment of diseases, such as vascular and ocular diseases.

### BACKGROUND OF THE INVENTION

Many surgical interventions require the placement of a medical device into the body. One prevalent surgical intervention often requiring such a device is percutaneous transluminal coronary angioplasty (“PTCA”). Many individuals suffer from circulatory disease caused by a progressive blockage of the blood vessels, which often leads to hypertension, ischemic injury, stroke, or myocardial infarction. Percutaneous transluminal coronary angioplasty is a medical procedure performed to increase blood flow through a damaged artery and is now the predominant treatment for coronary vessel stenosis. The increasing use of this procedure is attributable to its relatively high success rate and its minimal invasiveness compared with coronary bypass surgery. A limitation associated with PTCA is the abrupt closure of the vessel which can occur soon after angioplasty. Insertion of small spring-like medical devices called stents into such damaged vessels has proved to be a better approach to keep the vessels open as compared to systemic pharmacologic therapy.

While often necessary and beneficial for treating a variety of medical conditions, metal or polymeric devices (e.g., stents, catheters . . . ), after placement in the body, can give rise to numerous physiological complications. Some of these complications include: increased risk of infection; initiation of a foreign body response resulting in inflammation and fibrous encapsulation; and initiation of a detrimental wound healing response resulting in hyperplasia and restenosis. These problems have been particularly acute with the placement of stents in damaged arteries after angioplasty.

One promising approach is to provide the device with the ability to deliver bioactive agents in the vicinity of the implant. By doing so, some of the harmful effects associated with the implantation of medical devices can be diminished. Thus, for example, antibiotics can be released from the surface of the device to minimize the possibility of infection, and antiproliferative drugs can be released to inhibit hyperplasia. Another benefit to the local release of bioactive agents is the avoidance of toxic concentrations of drugs encountered when given systemically at sufficiently high doses to achieve therapeutic concentrations at the site where they are needed.

Although the potential benefit from using such bioactive agent-releasing medical devices is great, development of such medical devices has been slow. Progress has been hampered by many challenges, including: 1) the requirement, in some instances, for long term (i.e., at least several weeks) release of bioactive agents; 2) the need for a biocompatible, non-inflammatory device surface; 3) the demand for significant durability (and particularly, resistance to delamination and cracking), particularly with devices that undergo flexion and/or expansion when being implanted or used in the body; 4) concerns regarding the ability of the device to be manufactured in an economically viable and reproducible manner; and 5) the requirement that the finished device can be sterilized using conventional methods.

Implantable medical devices capable of delivering medicinal agents from hydrophobic polymer coatings have been described. See, for instance, U.S. Pat. No. 6,214,901; U.S. Pat. No. 6,344,035; U.S. Publication No. 2002-0032434; U.S. Publication No. 2002-0188037; U.S. Publication No. 2003-0031780; U.S. Publication No. 2003-0232087; U.S. Publication No. 2003-0232122; PCT Publication No. WO 99/55396; PCT Publication No. WO 03/105920; PCT Publication No. WO 03/105918; PCT Publication No. WO 03/105919 which collectively disclose, inter alia, coating compositions having a bioactive agent in combination with a polymer component such as polyalkyl(meth)acrylate or aromatic poly(meth)acrylate polymer and another polymer component such as poly(ethylene-co-vinyl acetate) for use in coating device surfaces to control and/or improve their ability to release bioactive agents in aqueous systems.

### SUMMARY OF THE INVENTION

The present invention provides a coating composition, and related methods for preparing and using the coating composition to coat a surface with a bioactive agent, for instance to coat the surface of an implantable medical device in a manner that permits the surface to release the bioactive agent over time when implanted in vivo.

The coating composition of this invention comprises one or more bioactive agents in combination with a plurality of polymers, including: (a) a first polymer component comprising a polymer selected from the group consisting of (i) ethylene copolymers with other alkylenes, (ii) polybutenes, (iii) aromatic group-containing copolymers, (iv) epichlorohydrin-containing polymers, (v) poly(alkylene-co-alkyl(meth)acrylates), and (vi) diolefin-derived, non-aromatic polymers and copolymers; and (b) a second polymer component comprising one or more polymers selected from the group consisting of poly(alkyl(meth)acrylates) and poly(aromatic(meth)acrylates), where “(meth)” will be understood by those skilled in the art to include such molecules in either the acrylic and/or methacrylic form (corresponding to the acrylates and/or methacrylates, respectively).

Applicants have discovered a group of first polymers that when used in combination with one or more second polymers can each meet or exceed the variety of criteria required of a composition of this invention, including in terms of its formulation, delivery, and/or coated characteristics.

In various embodiments, with regard to its formulation, a coating composition of this invention may be provided in the form of a true solution by the use of one or more solvents. Such solvents, in turn, are not only capable of dissolving the polymers and bioactive agent in solution, as compared to dispersion or emulsion, but they are also sufficiently volatile to permit the composition to be effectively applied to a surface (e.g., as by spraying) and quickly removed (e.g., as by drying) to provide a stable and desirable coated composition. In turn, the coated composition is itself homogeneous, with the first and second polymers effectively serving as cosolvents for each other, and bioactive agent substantially equally sequestered within them both.

In some embodiments, the ability to form a true solution using the claimed polymer combinations is desirable when considering the inclusion of potentially significant amounts of bioactive agent with the polymer blend. In various embodiments of the present invention, the coating composition is not only in the form of a true solution, but one in which bioactive agent is present at saturated or supersaturated levels. Without intending to be bound by theory, it appears that it is by virtue of the ability to achieve such solutions, that release of the bioactive agent from the coated composition is best accomplished and facilitated. In turn, it appears that the release of bioactive agent from such a system is due, at least in part, to its inherent instability within the coated composition itself, coupled with its physical/chemical preference for surrounding tissues and fluids. In turn, those skilled in the art will appreciate the manner in which the various ingredients and amounts in a composition of this invention can be adjusted to provide desired release kinetics and for any particular bioactive agent, solvent and polymer combination.

With regard to its delivery, various embodiments including a composition of this invention meets or exceeds further criteria in its ability to be sterilized, stored, and delivered to a surface in a manner that preserves its desired characteristics, yet using conventional delivery means, such as spraying. In some embodiments, such delivery involves spraying the composition onto a device surface in a manner that avoids or minimizes phase separation of the polymer components.

Finally, and with regard to its coated characteristics, a composition of this invention permits polymer ratios to be varied in a manner that provides not only an optimal combination of such attributes as biocompatibility, durability, and bioactive agent release kinetics, but also, in some embodiments, provides a coated composition that is homogeneous, and hence substantially optically clear upon microscopic examination. Even more surprisingly, in some embodiments, the compositions of this invention will provide these and other features, with or without optional pretreatment of a metallic surface. The ability to achieve or exceed any of these criteria, let alone most if not all of them, was not expected.

In turn, compositions of the present invention provide properties that are comparable or better than those obtained with previous polymer blend compositions. This, in turn, provides a variety of new and further opportunities, including with respect to both the type and concentration of bioactive agents that can be coated, as well as the variety of medical devices, and surfaces, themselves. In turn, the present invention also provides a combination that includes a medical device coated with a composition of this invention, as well as a method of preparing and using such a combination.

### BRIEF DESCRIPTION OF THE FIGURES

FIG. 1 depicts a graph illustrating the cumulative bioactive agent release profiles for coating compositions according to the present invention applied to stents, as described in Example 1.

FIG. 2 depicts a graph illustrating the cumulative bioactive agent release profiles for coating compositions according to the present invention applied to stents, as described in Example 2.

FIG. 3 depicts a graph illustrating the cumulative bioactive agent release profiles for coating compositions according to the present invention applied to stents, as described in Example 3.

FIG. 4 depicts a graph illustrating the cumulative bioactive agent release profiles for coating compositions according to the present invention applied to stents, as described in Example 4.

FIG. 5 depicts a graph illustrating the cumulative bioactive agent release profiles for coating compositions according to the present invention applied to stents, as described in Example 5.

FIG. 6 depicts a graph illustrating the cumulative bioactive agent release profiles for coating compositions according to the present invention applied to stents, as described in Example 6.

FIG. 7 depicts a graph illustrating the stress/strain measurements of first polymer components used in coating compositions according to the present invention, as described in Example 8.

FIG. 8 depicts a 100 micron wide and 10 micron deep Raman image taken by measuring the Raman intensity at 2900 cm⁻¹ of a coating composition according to the present invention.

FIG. 9 depicts a 100 micron wide and 10 micron deep Raman image taken by measuring the Raman intensity at 1630 cm⁻¹ for the same region of stent coating shown in FIG. 9.

FIG. 10 depicts a graph illustrating the cumulative bioactive agent release profiles for coating compositions according to the present invention applied to stents, as described in Example 10.

FIG. 10A depicts a bar chart illustrating the durability profiles for coating compositions according to the present invention applied to stents, as described in Example 10.

FIG. 11 depicts a graph illustrating the cumulative bioactive agent release profiles for coating compositions according to the present invention applied to stents, as described in Example 11.

FIG. 12 depicts a graph illustrating the cumulative bioactive agent release profiles for coating compositions according to the present invention applied to stents, as described in Example 12.

FIG. 13 depicts a scanning electron microscope image a coated stent including a coating composition according to the present invention after conventional crimping and balloon expansion procedures.

FIG. 14 depicts a graph illustrating the cumulative bioactive agent release profiles for coating compositions according to the present invention applied to stents, as described in Example 15.

FIG. 15 depicts a graph illustrating the cumulative bioactive agent release profiles for coating compositions according to the present invention applied to stents, as described in Example 15.

FIG. 16 depicts a graph illustrating the cumulative bioactive agent release profiles for coating compositions and topcoats according to the present invention applied to stents, as described in Example 16.

FIG. 17 shows a medical device as described in Example 17.

FIG. 18 shows a medical device as described in Example 17.

FIG. 19A shows a plot of data as described in Example 17.

FIG. 19B shows the plot of FIG. 19A with tilt and curvature correction.

FIG. 20A shows a surface plot of a roughness test as described in Example 17.

FIG. 20B shows a 3D representation of FIG. 20A.

FIG. 21A shows a surface plot of a roughness test as described in Example 17.

FIG. 21B shows a 3D representation of FIG. 21A.

### DETAILED DESCRIPTION

Without intending to be bound by theory, it appears that suitable first polymers for use in a composition of this invention provide an optimal combination of such properties as glass transition temperature (T_{g}) and diffusion constant for the particular bioactive agent of choice. Along with melting temperature (Tₘ), T_{g} is an important parameter of a given polymer (including copolymer), and particularly amorphous polymers, that can be used to characterize its properties over a wide temperature range. A polymer is typically brittle at temperatures below its T_{g}, and flexible at temperatures above. Both Tₘ and T_{g} can be affected by such things as polymer structure and backbone flexibility, molecular weight, attractive forces, and pressure. For random copolymers and compatible polymer blends, only a single T_{g} is observed, usually lying intermediate between the T_{g} of the corresponding pure homopolymers. Different T_{g}'s are exhibited for incompatible polymer blends, and between the microdomains of block copolymers with mutually incompatible blocks. T_{g} can be measured by any suitable technique, e.g., dilatometry, refractive index, differential scanning calorimetry, dynamic mechanical measurement, and dielectric measurement.

Various second polymers (e.g., poly(n-butyl methacrylate)) of the present composition generally provide a T_{g} in the range of room to body temperature (e.g., from about 20° C. to about 40° C.), and hence tend to be somewhat stiffer polymers, in turn, providing a slower diffusion constant for a number of bioactive agents. Applicants have discovered the manner in which certain new polymers can be used as a first polymer component, to essentially balance, or temper the desired properties of the second polymer. Such first polymers will generally provide a lower glass transition temperature (e.g., below room temperature, and in some embodiments in the range of about 0° C. or less), together with a relatively high diffusion constant for the bioactive agent. By appropriately combining the two polymers with bioactive agent, those skilled in the art, given the present description, will be able to vary both the selection and ratios of first and second polymers, in order to determine an optimal combination of physical and mechanical properties, including bioactive agent diffusion and release kinetics, as well as durability and tenacity of the coating itself upon a particular surface, that best fits their particular needs.

Hence embodiments of the first polymer of this invention will generally provide an optimal combination of glass transition temperature (e.g., at or lower than that of the second polymer), compatibility with the bioactive agent of choice, acceptable solubility in the solvents of choice, as well as commercial availability and cost.

The term “coating composition”, as used herein, will refer to one or more vehicles (e.g., solutions, mixtures, emulsions, dispersions, blends, etc.) used to effectively coat a surface with bioactive agent, first polymer component and/or second polymer component, either individually or in any suitable combination.

The term “coated composition” will refer to the effective combination, upon the surface of a device, of bioactive agent, first polymer component and second polymer component, whether formed as the result of one or more coating vehicles or in one or more layers and/or steps.

Unless defined otherwise, the term “coating” will refer to the effective combination of bioactive agent, first polymer component and second polymer component, independent of the device surface, and whether formed as the result of one or more coating vehicles or in one or more layers.

Unless otherwise indicated, the term “molecular weight” and all polymeric molecular weights described herein are “weight average” molecular weights (“Mw”). As used herein “weight average molecular weight” or M_{w}, is an absolute method of measuring molecular weight and is particularly useful for measuring the molecular weight of a polymer preparation. The weight average molecular weight (M_{w}) can be defined by the following formula:

$${M}_{w} = \frac{{\sum }_{i} {N}_{i} {M}_{i}^{2}}{{\sum }_{i} {N}_{i} {M}_{i}}$$

wherein N represents the number of moles of a polymer in the sample with a mass of M, and Σᵢ is the sum of all NᵢMᵢ (species) in a preparation. The M_{w} can be measured using common techniques, such as light scattering or ultracentrifugation. Discussion of M_{w} and other terms used to define the molecular weight of polymer preparations can be found in, for example, Allcock, H. R. and Lampe, F. W., Contemporary Polymer Chemistry; pg 271 (1990).

As described and exemplified herein, a resultant composition can be coated using a plurality of individual steps or layers, including for instance, an initial layer having only bioactive agent (or bioactive agent with one or both of the polymer components), over which are coated one or more additional layers containing suitable combinations of bioactive agent, first polymer component and/or second polymer component, the combined result of which is to provide a coated composition of the invention. In turn, and in various embodiments, the invention further provides a method of reproducibly controlling the release (e.g., elution) of a bioactive agent from the surface of a medical device implanted in vivo. Those skilled in the art will appreciate the manner in which the combined effect of these various layers can be used and optimized to achieve various effects in vivo. In addition, the surface to which the composition is applied can itself be pretreated in a manner sufficient to improve attachment of the composition to the underlying (e.g., metallic) surface. Examples of such pretreatments include the use of compositions such as Parylene™ coatings, as described herein. Additional examples of such pretreatments include silane coupling agents, photografted polymers, epoxy primers, polycarboxylate resins, and physical roughening of the surface. It is further noted that the pretreatment compositions may be used in combination with each other or may be applied in separate layers to form a pretreatment coating on the surface of the medical device.

While not intending to be bound by theory, the release kinetics of the bioactive agent in vivo are thought to generally include both a short term (“burst”) release component, within the order of minutes to hours after implantation, and a longer term release component, which can range from on the order of hours to days or even months or years of useful release.

Additionally, the ability to coat a device in the manner of the present invention provides greater latitude in the composition of various coating layers, e.g., permitting more or less of the second polymer component (i.e., poly(alkyl(meth)acrylate) and/or poly(aromatic(meth)acrylate)) to be used in coating compositions used to form different layers (e.g., as a topcoat layer). This, in turn, provides the opportunity to further control release and elution of the bioactive agent from the overall coating.

The coating composition and method can be used to control the amount and rate of bioactive agent (e.g., drug) release from one or more surfaces of implantable medical devices. In various embodiments, the method employs a mixture of hydrophobic polymers in combination with one or more bioactive agents, such as a pharmaceutical agent, such that the amount and rate of release of agent(s) from the medical device can be controlled, e.g., by adjusting the relative types and/or concentrations of hydrophobic polymers in the mixture. For a given combination of polymers, for instance, this approach permits the release rate to be adjusted and controlled by simply adjusting the relative concentrations of the polymers in the coating mixture. This provides an additional means to control rate of bioactive agent release besides the conventional approach of varying the concentration of bioactive agent in a coated composition.

Some embodiments of the invention include a method of coating a device comprising the step of applying the composition to the device surface under conditions of controlled relative humidity (at a given temperature), for instance, under conditions of increased or decreased relative humidity as compared to ambient humidity. Humidity can be “controlled” in any suitable manner, including at the time of preparing and/or using (as by applying) the composition, for instance, by coating the surface in a confined chamber or area adapted to provide a relative humidity different than ambient conditions, and/or by adjusting the water content of the coating or coated composition itself. Without intending to be bound by theory, it appears that the elution rate of a bioactive agent from a coating composition generally increases as relative humidity increases.

In various embodiments, the coating composition of this invention includes a mixture of two or more polymers having complementary physical characteristics, and a bioactive agent or agents applicable to the surface of an implantable medical device. The device can be of any suitable type or configuration, and in some embodiments, is one that undergoes flexion and/or expansion upon implantation or use, as in the manner of a stent or catheter. The applied coating composition is cured (e.g., by solvent evaporation) to provide a tenacious and flexible bioactive-releasing composition on the surface of the medical device. Such coating compositions are particularly well suited for devices that are themselves sufficiently small, or have portions that are sufficiently small (as in the struts of an expandable stent or the twists of an ocular coil), to permit the coated composition to form a contiguous, e.g., circumferential, coating, thereby further improving the ability of the coating to remain intact (e.g., avoid delamination).

The complementary polymers are selected such that a broad range of relative polymer concentrations can be used without detrimentally affecting the desirable physical characteristics of the polymers. By use of the polymer combinations (including mixtures and blends) of the invention the bioactive release rate from a coated medical device can be manipulated by adjusting the relative concentrations of the polymers.

In additional embodiments, the present invention relates to a coating composition and related method for coating an implantable medical device which undergoes flexion and/or expansion upon implantation. However it is noted that the coating composition may also be utilized with medical devices that have minimal or do not undergo flexion and/or expansion. The structure and composition of the underlying device can be of any suitable, and medically acceptable, design and can be made of any suitable material that is compatible with the coating itself. The natural or pretreated surface of the medical device is provided with a coating containing one or more bioactive agents.

A first polymer component of this invention provides an optimal combination of similar properties, and particularly when used in admixture with the second polymer component. In some embodiments, a first polymer is a polymer selected from the group consisting of (i) ethylene copolymers with other alkylenes, (ii) polybutenes, (iii) aromatic group-containing copolymers, (iv) epichlorohydrin-containing polymers (v) poly(alkylene-co-alkyl(meth)acrylates), and (vi) diolefin-derived, non-aromatic polymers and copolymers.

Examples of suitable first polymers are commercially available from sources such as Sigma-Aldrich.

A first polymer component may be selected from one or more ethylene copolymers with other alkylenes. Various first polymers for use in this invention comprise ethylene copolymers with other alkylenes, which in turn, can include straight chain and branched alkylenes, as well as substituted or unsubstituted alkylenes. Examples include copolymers prepared from alkylenes that comprise from 3 to 8 branched or linear carbon atoms, inclusive, in various embodiments, alkylene groups that comprise from 3 to 4 branched or linear carbon atoms, inclusive, and in some embodiments, the alkylene group contains 3 carbon atoms (e.g., propylene). In some embodiments, the other alkylene is a straight chain alkylene (e.g., 1-alkylene).

Various copolymers of this type can comprise from about 20% to about 90% (based on moles) of ethylene, and in some embodiments, from about 35% to about 80% (mole) of ethylene. Such copolymers will have a molecular weight of between about 30 kilodaltons to about 500 kilodaltons. Examples of such copolymers are selected from the group consisting of poly(ethylene-co-propylene), poly(ethylene-co-1-butene), polyethylene-co-1-butene-co-1-hexene) and/or poly(ethylene-co-1-octene).

Examples of particular copolymers include poly(ethylene-co-propylene) random copolymers in which the copolymer contains from about 35% to about 65% (mole) of ethylene; and in some embodiments, from about 55% to about 65% (mole) ethylene, and the molecular weight of the copolymer is from about 50 kilodaltons to about 250 kilodaltons, in some embodiments from about 100 kilodaltons to about 200 kilodaltons.

Copolymers of this type can optionally be provided in the form of random terpolymers prepared by the polymerization of both ethylene and propylene with optionally one or more additional diene monomers, such as those selected from the group consisting of ethylidene norborane, dicyclopentadiene and/or hexadiene. Various terpolymers of this type can include up to about 5% (mole) of the third diene monomer.

Other examples of suitable copolymers of this type are commercially available from sources such as Sigma-Aldrich and include the following products. For example, suitable copolymers of this type and their related descriptions may be found in the 2003-2004 Aldrich Handbook of Fine Chemicals and Laboratory Equipment, the entire contents of which are incorporated by reference herein. Examples of such copolymers include, but are not limited to poly(ethylene-co-propylene), poly(ethylene-co-1-butene), poly(ethylene-co-1-butene-co-1-hexene), poly(ethylene-co-1-octene) and poly(ethylene-co-propylene-co-5-methylene-2-norborene).

Alternatively, a first polymer component may be selected from one or more polybutenes. “Polybutenes” suitable for use in the present invention include polymers derived by homopolymerizing or randomly interpolymerizing isobutylene, 1-butene and/or 2-butene. The polybutene can be a homopolymer of any of the isomers or it can be a copolymer or a terpolymer of any of the monomers in any ratio. In various embodiments, the polybutene contains at least about 90% (wt) of isobutylene or 1-butene, and in some embodiments, the polybutene contains at least about 90% (wt) of isobutylene. The polybutene may contain non-interfering amounts of other ingredients or additives, for instance it can contain up to 1000 ppm of an antioxidant (e.g., 2,6-di-tert-butyl-methylphenol).

In various embodiments, the polybutene has a molecular weight between about 100 kilodaltons and about 1,000 kilodaltons, in some embodiments, between about 150 kilodaltons and about 600 kilodaltons, and in some embodiments, between about 150 kilodaltons and about 250 kilodaltons. In other embodiments, the polybutene has a molecular weight between about 150 kilodaltons and about 1,000 kilodaltons, optionally, between about 200 kilodaltons and about 600 kilodaltons, and further optionally, between about 350 kilodaltons and about 500 kilodaltons. Polybutenes having a molecular weight greater than about 600 kilodaltons, including greater than 1,000 kilodaltons are available but are expected to be more difficult to work with. Other examples of suitable copolymers of this type are commercially available from sources such as Sigma-Aldrich.

Additional alternative first polymers include aromatic group-containing copolymers, including random copolymers, block copolymers and graft copolymers. In various embodiments, the aromatic group is incorporated into the copolymer via the polymerization of styrene, and in some embodiments, the random copolymer is a copolymer derived from copolymerization of styrene monomer and one or more monomers selected from butadiene, isoprene, acrylonitrile, a C₁-C₄ alkyl(meth)acrylate (e.g., methyl methacrylate) and/or butene (e.g., isobutylene). Useful block copolymers include copolymer containing (a) blocks of polystyrene, (b) blocks of a polyolefin selected from polybutadiene, polyisoprene and/or polybutene (e.g., polyisobutylene), and (c) optionally a third monomer (e.g., ethylene) copolymerized in the polyolefin block.

The aromatic group-containing copolymers may contain about 10% to about 50% (wt) of polymerized aromatic monomer and the molecular weight of the copolymer may be from about 50 kilodaltons to about 500 kilodaltons. In some embodiments, the molecular weight of the copolymer may be from about 300 kilodaltons to about 500 kilodaltons. In other embodiments, the molecular weight of the copolymer may be from about I 00 kilodaltons to about 300 kilodaltons.

Other examples of suitable copolymers of this type are commercially available from sources such as Sigma-Aldrich and include, but are not limited to, poly(styrene-co-butadiene) (random), polystyrene-block-polybutadiene, polystyrene-block-polybutadiene-block-polystyrene, polystyrene-block-poly(ethylene-ran-butylene)-block-polystyrene, polystyrene-block-polyisoprene-block-polystyrene, polystyrene-block-polyisobutylene-block-polystyrene, poly(styrene-co-acrylonitrile), poly(styrene-co-butadiene-co-acrylonitrile) and poly(styrene-co-butadiene-co-methyl methacrylate).

Additional alternative first polymers include epichlorohydrin homopolymers and poly(epichlorohydrin-co-alkylene oxide)copolymers. In some embodiments, in the case of the copolymer, the copolymerized alkylene oxide is ethylene oxide. In various embodiments, epichlorohydrin content of the epichlorohydrin-containing polymer is from about 30% to 100% (wt), and in some embodiments from about 50% to 100% (wt). In some embodiments, the epichlorohydrin-containing polymers have a Mw from about 100 kilodaltons to about 300 kilodaltons.

Other examples of suitable copolymers of this type are commercially available from sources such as Sigma-Aldrich and include, but are not limited to, polyepichlorohydrin and poly(epichlorohydrin-co-ethylene oxide).

As another example, a first polymer component may be selected from one or more poly(alkylene-co-alkyl(meth)acrylates. Various poly(alkylene-co-alkyl(meth)acrylates) include those copolymers in which the alkyl groups are either linear or branched, and substituted or unsubstituted with non-interfering groups or atoms. In various embodiments, such alkyl groups comprise from 1 to 8 carbon atoms, inclusive, and in some embodiments, from 1 to 4 carbon atoms, inclusive. In one example, the alkyl group is methyl.

In various embodiments, copolymers that include such alkyl groups comprising from about 15% to about 80% (wt) of alkyl acrylate. When the alkyl group is methyl, the polymer may contain from about 20% to about 40% methyl acrylate, and in some embodiments from about 25 to about 30% methyl acrylate. When the alkyl group is ethyl, the polymer, in some embodiments, contains from about 15% to about 40% ethyl acrylate, and when the alkyl group is butyl, the polymer, in some embodiments, contains from about 20% to about 40% butyl acrylate.

The alkylene groups are selected from ethylene and/or propylene, and more in various embodiments, the alkylene group is ethylene. In various embodiments, the (meth)acrylate comprises an acrylate (i.e., no methyl substitution on the acrylate group). Various copolymers provide a molecular weight (Mw) of about 50 kilodaltons to about 500 kilodaltons, and in some embodiments, Mw is 50 kilodaltons to about 200 kilodaltons.

The glass transition temperature for these copolymers varies with ethylene content, alkyl length on the (meth)acrylate and whether the first copolymer is an acrylate or methacrylate. At higher ethylene content, the glass transition temperature tends to be lower, and closer to that of pure polyethylene (−120° C.). A longer alkyl chain also lowers the glass transition temperature. A methyl acrylate homopolymer has a glass transition temperature of about 10° C. while a butyl acrylate homopolymer has one of −54° C.

Copolymers such as poly(ethylene-co-methyl acrylate), poly(ethylene-co-butyl acrylate) and poly(ethylene-co-2-ethylhexyl acrylate)copolymers are available commercially from sources such as Atofina Chemicals, Inc., Philadelphia, Pa., and can be prepared using methods available to those skilled in the respective art.

Other examples of suitable polymers of this type are commercially available from sources such as Sigma-Aldrich and include, but are not limited to, poly(ethylene-co-methyl acrylate), poly(ethylene-co-ethyl acrylate), and poly(ethylene-co-butyl acrylate).

First polymers may also include diolefin-derived, non-aromatic polymers and copolymers, including those in which the diolefin monomer used to prepare the polymer or copolymer is selected from butadiene (CH₂═CH—CH═CH₂) and/or isoprene (CH₂═CH—C(CH₃)═CH₂). A butadiene polymer can include one or more butadiene monomer units which can be selected from the monomeric unit structures (a), (b), or (c): 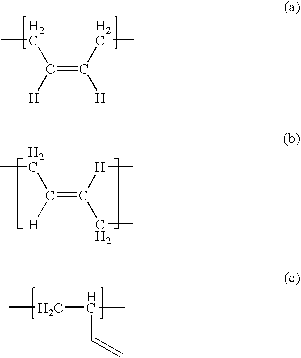
An isoprene polymer can include one or more isoprene monomer units which can be selected from the monomeric unit structures (d), (e), (f) or (g):

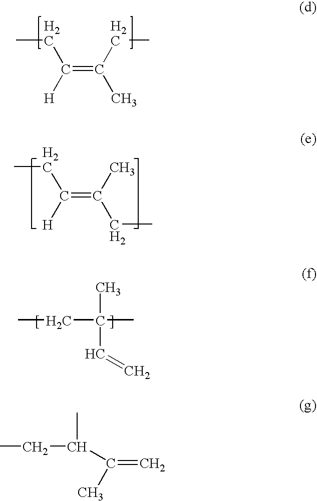

In some embodiments, the polymer is a homopolymer derived from diolefin monomers or is a copolymer of diolefin monomer with non-aromatic mono-olefin monomer, and optionally, the homopolymer or copolymer can be partially hydrogenated. Such polymers can be selected from the group consisting of polybutadienes containing polymerized cis-, trans- and/or 1,2-monomer units, and in some embodiments, a mixture of all three co-polymerized monomer units, and polyisoprenes containing polymerized cis-1,4- and/or trans-1,4-monomer units, polymerized 1,2-vinyl monomer units, polymerized 3,4-vinyl monomer units and/or others as described in the Encyclopedia of Chemical Technology, Vol. 8, page 915 (1993), the entire contents of which is hereby incorporated by reference.

Alternatively, the first polymer is a copolymer, including graft copolymers, and random copolymers based on a non-aromatic mono-olefin co-monomer such as acrylonitrile, an alkyl(meth)acrylate and/or isobutylene. In various embodiments, when the mono-olefin monomer is acrylonitrile, the interpolymerized acrylonitrile is present at up to about 50% by weight; and when the mono-olefin monomer is isobutylene, the diolefin monomer is isoprene (e.g., to form what is commercially known as a “butyl rubber”). In some embodiments, the polymers and copolymers have a Mw between about 50 kilodaltons and about 1,000 kilodaltons. In other embodiments, the polymers and copolymers have a Mw between about 100 kilodaltons and about 450 kilodaltons. In yet other embodiments the polymers and copolymers have a Mw between about 150 kilodaltons and about 1,000 kilodaltons, and optionally between about 200 kilodaltons and about 600 kilodaltons.

Other examples of suitable first polymers of this type are commercially available from sources such as Sigma-Aldrich, and include, but are not limited to, polybutadiene, poly(butadiene-co-acrylonitrile), polybutadiene-block-polyisoprene, polybutadiene-graft-poly(methyl acrylate-co-acrylonitrile), polyisoprene, and partially hydrogenated polyisoprene.

A second polymer component of this invention provides an optimal combination of various structural/functional properties, including hydrophobicity, durability, bioactive agent release characteristics, biocompatibility, molecular weight, and availability. In one such an embodiment, the composition comprises at least one second polymer component selected from the group consisting of poly(alkyl(meth)acrylates) and poly(aromatic (meth)acrylates).

In various embodiments, the second polymer component is a poly(alkyl)methacrylate, that is, an ester of a methacrylic acid. Examples of suitable poly(alkyl(meth)acrylates) include those with alkyl chain lengths from 2 to 8 carbons, inclusive, and with molecular weights from 50 kilodaltons to 900 kilodaltons. In various embodiments the polymer mixture includes a poly(alkyl(meth)acrylate) with a molecular weight of from about 100 kilodaltons to about 1000 kilodaltons, in some embodiments, from about 150 kilodaltons to about 500 kilodaltons, and in some embodiments from about 200 kilodaltons to-about 400 kilodaltons. An example of a particular second polymer is poly(n-butyl methacrylate). Examples of other polymers are poly(n-butyl methacrylate-co-methyl methacrylate, with a monomer ratio of 3:1, poly(n-butyl methacrylate-co-isobutyl methacrylate, with a monomer ratio of 1:1 and poly(t-butyl methacrylate). Such polymers are available commercially (e.g., from Sigma-Aldrich, Milwaukee, Wis.) with molecular weights ranging from about 150 kilodaltons to about 350 kilodaltons, and with varying inherent viscosities, solubilities and forms (e.g., as slabs, granules, beads, crystals or powder).

Examples of suitable poly(aromatic(meth)acrylates) include poly(aryl(meth)acrylates), poly(aralkyl(meth)acrylates), poly(alkaryl(meth)acrylates), poly(aryloxyalkyl(meth)acrylates), and poly(alkoxyaryl(meth)acrylates). Such terms are used to describe polymeric structures wherein at least one carbon chain and at least one aromatic ring are combined with (meth)acrylic groups, typically esters, to provide a composition of this invention. For instance, and more specifically, a poly(aralkyl (meth)acrylate) can be made from aromatic esters derived from alcohols also containing aromatic moieties, such as benzyl alcohol. Similarly, a poly(alkaryl(meth)acrylate) can be made from aromatic esters derived from aromatic alcohols such as p-anisole. Suitable poly(aromatic(meth)acrylates) include aryl groups having from 6 to 16 carbon atoms and with molecular weights from about 50 to about 900 kilodaltons. Examples of suitable poly(aryl(meth)acrylates) include poly(9-anthracenyl methacrylate), poly(chlorophenyl acrylate), poly(methacryloxy-2-hydroxybenzophenone), poly(methacryloxybenzotriazole), poly(naphthyl acrylate), poly(naphthylmethacrylate), poly-4-nitrophenylacrylate, poly(pentachloro(bromo, fluoro)acrylate) and methacrylate, poly(phenyl acrylate) and poly(phenyl methacrylate). Examples of suitable poly(aralkyl(meth)acrylates) include poly(benzyl acrylate), poly(benzyl methacrylate), poly(2-phenethyl acrylate), poly(2-phenethyl methacrylate) and poly(1-pyrenylmethyl methacrylate). Examples of suitable poly(alkaryl(meth)acrylates include poly(4-sec-butylphenyl methacrylate), poly(3-ethylphenyl acrylate), and poly(2-methyl-1-naphthyl methacrylate). Examples of suitable poly(aryloxyalkyl(meth)acrylates) include poly(phenoxyethyl acrylate), poly(phenoxyethyl methacrylate), and poly(polyethylene glycol phenyl ether acrylate) and poly(polyethylene glycol phenyl ether methacrylate) with varying polyethylene glycol molecular weights. Examples of suitable poly(alkoxyaryl(meth)acrylates) include poly(4-methoxyphenyl methacrylate), poly(2-ethoxyphenyl acrylate) and poly(2-methoxynaphthyl acrylate).

Acrylate or methacrylate monomers or polymers and/or their parent alcohols are commercially available from Sigma-Aldrich (Milwaukee, Wis.) or from Polysciences, Inc, (Warrington, Pa.).

Optionally, the coating composition may include one or more additional polymers in combination with the first and second polymer components, the additional polymers being, for example, selected from the group consisting of (i) poly(alkylene-co-alkyl(meth)acrylates, (ii) ethylene copolymers with other alkylenes, (iii) polybutenes, (iv) diolefin-derived, non-aromatic polymers and copolymers, (v) aromatic group-containing copolymers, (vi) epichlorohydrin-containing polymers, including each as disclosed and described above in the sections describing first polymers, and (vii) poly(ethylene-co-vinyl acetate). Generally, if one or more additional polymers are included, the one or more additional polymers are different from the first polymer component used in the coating composition. In some embodiments, the additional polymers may substitute up to about 25% of the first polymer. In other embodiments, the additional polymers may substitute up to about 50% of the first polymer.

As discussed above, a suitable additional polymer that may be utilized in the coating composition of the present invention includes poly(ethylene-co-vinyl acetate) (pEVA). Examples of suitable polymers of this type are available commercially and include poly(ethylene-co-vinyl acetate) having vinyl acetate concentrations of from about 8% and about 90%, in some embodiments, from about 20 to about 40 weight percent and in some embodiments, from about 30 to about 34 weight percent. Such polymers are generally found in the form of beads, pellets, granules, etc. It has generally been found that pEVA co-polymers with lower percent vinyl acetate become increasingly insoluble in typical solvents.

In some embodiments, coating compositions for use in this invention includes mixtures of first and second polymer components as described herein. Optionally, both first and second polymer components are purified for such use to a desired extent and/or provided in a form suitable for in vivo use. Moreover, biocompatible additives may be added, such as dyes and pigments (e.g., titanium dioxide, Solvent Red 24, iron oxide, and Ultramarine Blue); slip agents (e.g., amides such as oleyl palmitamide, N,N′-ethylene bisoleamide, erucamide, stearamide, and oleamide); antioxidants (e.g. butylated hydroxytoluene (BHT), vitamin E (tocopherol), BNX™, dilauryl thiodipropionate (DLTDP), IrganoX™ series, phenolic and hindered phenolic antioxidants, organophosphites (e.g., trisnonylphenyl phosphite, Irgafos™ 168), lactones (e.g., substituted benzofuranone), hydroxylamine, and MEHQ (monomethyl ether of hydroquinone)); surfactants (e.g., anionic fatty acid surfactants (e.g., sodium lauryl sulfate, sodium dodecylbenzenesulfonate, sodium stearate, and sodium palmitate), cationic fatty acid surfactants (e.g., quaternary ammonium salts and amine salts), and nonionic ethoxylated surfactants (e.g., ethoxylated p-octylphenol)); and leachable materials (i.e., permeation enhancers) (e.g., hydrophilic polymers (e.g., poly(ethylene glycol), polyvinylpyrrolidone, and poly(vinyl alcohol)) and hydrophilic small molecules (e.g., sodium chloride, glucose)). In addition, any impurities may be removed by conventional methods available to those skilled in the art.

In various embodiments, the polymer mixture includes a first polymer component comprising one or more polymers selected from the group consisting of (i) ethylene copolymers with other alkylenes, (ii) polybutenes, (iii) aromatic group-containing copolymers, (iv) epichlorohydrin-containing polymers, (v) poly(alkylene-co-alkyl(meth)acrylates), and (vi) diolefin-derived, non-aromatic polymers and copolymers, and a second polymer component selected from the group consisting of poly (alkyl(meth)acrylates) and poly(aromatic(meth)acrylates) and having a molecular weight of from about 150 kilodaltons to about 500 kilodaltons, and in some embodiments from about 200 kilodaltons to about 400 kilodaltons.

These mixtures of polymers have proven useful with absolute polymer concentrations (i.e., the total combined concentrations of both polymers in the coating composition), of between about 0.1 and about 50 percent (by weight), and in some embodiments, between about 0.1 and about 35 percent (by weight). Various polymer mixtures contain at least about 10 percent by weight of either the first polymer or the second polymer.

In some embodiments, the polymer composition may comprise about 5% to about 95% of the first and/or second polymers based on the total weights of the first and second polymers. In a another group of embodiments, the composition may comprise about 15% to about 85% of the first and/or second polymers. In some embodiments, the composition may include about 25% to about 75% of the first and/or second polymers.

In various embodiments, the bioactive agent may comprise about 1% to about 75% of the first polymer, second polymer, and bioactive agent mixture (i.e., excluding solvents and other additives). In some embodiments, the bioactive agent may comprise about 5% to about 60% of such a mixture. In some embodiments, the bioactive agent may comprise about 25% to about 45% of such a mixture. The concentration of the bioactive agent or agents dissolved or suspended in the coating mixture can range from about 0.01 to about 90 percent, by weight, based on the weight of the final coating composition, and in some embodiments, from about 0.1 to about 50 percent by weight.

The term “bioactive agent” and “active agent”, as used herein, will refer to a wide range of biologically active materials or drugs that can be incorporated into a coating composition of the present invention. In some embodiments of the present invention, the bioactive agent(s) to be incorporated do not chemically interact with the coating composition during fabrication or during the bioactive agent release process. The bioactive agents as described herein may also be included in one or more additional layers or coatings, such as, for example, a pretreatment coating and/or protective coating. In embodiments so provided, the bioactive agent in the coating composition may be the same as or different than the bioactive agent included in the pretreatment coating and/or protective coating. Further, such bioactive agents may sometimes be referred to herein as the “pretreatment coating bioactive agent” or the “protective coating bioactive agent.”

An amount of biologically active agent can be applied to the device to provide a therapeutically effective amount of the agent to a patient receiving the coated device. Particularly useful agents include those that affect cardiovascular function or that can be used to treat cardiovascular-related disorders. In an embodiment, the active agent includes estradiol. In an embodiment, the active agent includes rapamycin. In an embodiment, the active agent includes paclitaxel.

Active agents useful in the present invention can include many types of therapeutics including thrombin inhibitors, antithrombogenic agents, thrombolytic agents, fibrinolytic agents, anticoagulants, anti-platelet agents, vasospasm inhibitors, calcium channel blockers, steroids, vasodilators, anti-hypertensive agents, antimicrobial agents, antibiotics, antibacterial agents, antiparasite and/or antiprotozoal solutes, antiseptics, antifungals, angiogenic agents, anti-angiogenic agents, inhibitors of surface glycoprotein receptors, antimitotics, microtubule inhibitors, antisecretory agents, actin inhibitors, remodeling inhibitors, antisense nucleotides, anti-metabolites, miotic agents, antiproliferatives, anticancer chemotherapeutic agents, anti-neoplastic agents, antipolymerases, antivirals, anti-AIDS substances, anti-inflammatory steroids or non-steroidal anti-inflammatory agents, analgesics, antipyretics, immunosuppressive agents, immunomodulators, growth hormone antagonists, growth factors, radiotherapeutic agents, peptides, proteins, enzymes, extracellular matrix components, ACE inhibitors, free radical scavengers, chelators, anti-oxidants, photodynamic therapy agents, gene therapy agents, anesthetics, immunotoxins, neurotoxins, opioids, dopamine agonists, hypnotics, antihistamines, tranquilizers, anticonvulsants, muscle relaxants and anti-Parkinson substances, antispasmodics and muscle contractants, anticholinergics, ophthalmic agents, antiglaucoma solutes, prostaglandin, antidepressants, antipsychotic substances, neurotransmitters, anti-emetics, imaging agents, specific targeting agents, and cell response modifiers.

More specifically, in embodiments the active agent can include heparin, covalent heparin, synthetic heparin salts, or another thrombin inhibitor; hirudin, hirulog, argatroban, D-phenylalanyl-L-poly-L-arginyl chloromethyl ketone, or another antithrombogenic agent; urokinase, streptokinase, a tissue plasminogen activator, or another thrombolytic agent; a fibrinolytic agent; a vasospasm inhibitor; a calcium channel blocker, a nitrate, nitric oxide, a nitric oxide promoter, nitric oxide donors, dipyridamole, or another vasodilator; HYTRIN® or other antihypertensive agents; a glycoprotein IIb/IIIa inhibitor (abciximab) or another inhibitor of surface glycoprotein receptors; aspirin, ticlopidine, clopidogrel or another antiplatelet agent; colchicine or another antimitotic, or another microtubule inhibitor; dimethyl sulfoxide (DMSO), a retinoid, or another antisecretory agent; cytochalasin or another actin inhibitor; cell cycle inhibitors; remodeling inhibitors; deoxyribonucleic acid, an antisense nucleotide, or another agent for molecular genetic intervention; methotrexate, or another antimetabolite or antiproliferative agent; tamoxifen citrate, TAXOL®, paclitaxel, or the derivatives thereof, rapamycin (or other rapalogs), vinblastine, vincristine, vinorelbine, etoposide, tenopiside, dactinomycin (actinomycin D), daunorubicin, doxorubicin, idarubicin, anthracyclines, mitoxantrone, bleomycin, plicamycin (mithramycin), mitomycin, mechlorethamine, cyclophosphamide and its analogs, chlorambucil, ethylenimines, methylmelamines, alkyl sulfonates (e.g., busulfan), nitrosoureas (carmustine, etc.), streptozocin, methotrexate (used with many indications), fluorouracil, floxuridine, cytarabine, mercaptopurine, thioguanine, pentostatin, 2-chlorodeoxyadenosine, cisplatin, carboplatin, procarbazine, hydroxyurea, morpholino phosphorodiamidate oligomer or other anti-cancer chemotherapeutic agents; cyclosporin, tacrolimus (FK-506), pimecrolimus, azathioprine, mycophenolate mofetil, mTOR inhibitors, or another immunosuppressive agent; cortisol, cortisone, dexamethasone, dexamethasone sodium phosphate, dexamethasone acetate, dexamethasone derivatives, betamethasone, fludrocortisone, prednisone, prednisolone, 6U-methylprednisolone, triamcinolone (e.g., triamcinolone acetonide), or another steroidal agent; trapidil (a PDGF antagonist), angiopeptin (a growth hormone antagonist), angiogenin, a growth factor (such as vascular endothelial growth factor (VEGF)), or an anti-growth factor antibody (e.g., ranibizumab, which is sold under the tradename LUCENTIS®), or another growth factor antagonist or agonist; dopamine, bromocriptine mesylate, pergolide mesylate, or another dopamine agonist; ⁶Co (5.3 year half life), ¹⁹²Ir (73.8 days), ³²P (14.3 days), ¹¹¹In (68 hours), ⁹⁰Y (64 hours), ⁹⁹Tc (6 hours), or another radiotherapeutic agent; iodine-containing compounds, barium-containing compounds, gold, tantalum, platinum, tungsten or another heavy metal functioning as a radiopaque agent; a peptide, a protein, an extracellular matrix component, a cellular component or another biologic agent; captopril, enalapril or another angiotensin converting enzyme (ACE) inhibitor; angiotensin receptor blockers; enzyme inhibitors (including growth factor signal transduction kinase inhibitors); ascorbic acid, alpha tocopherol, superoxide dismutase, deferoxamine, a 21-aminosteroid (lasaroid) or another free radical scavenger, iron chelator or antioxidant; a ¹⁴C-, ³H-, ¹³H-, ³²P- or ³⁶S-radiolabelled form or other radiolabelled form of any of the foregoing; an estrogen (such as estradiol, estriol, estrone, and the like) or another sex hormone; AZT or other antipolymerases; acyclovir, famciclovir, rimantadine hydrochloride, ganciclovir sodium, Norvir, Crixivan, or other antiviral agents; 5-aminolevulinic acid, meta-tetrahydroxyphenylchlorin, hexadecafluorozinc phthalocyanine, tetramethyl hematoporphyrin, rhodamine 123 or other photodynamic therapy agents; an IgG2 Kappa antibody against Pseudomonas aeruginosa exotoxin A and reactive with A431 epidermoid carcinoma cells, monoclonal antibody against the noradrenergic enzyme dopamine beta-hydroxylase conjugated to saporin, or other antibody targeted therapy agents; gene therapy agents; enalapril and other prodrugs; PROSCAR®, HYTRIN® or other agents for treating benign prostatic hyperplasia (BHP); mitotane, aminoglutethimide, breveldin, acetaminophen, etodalac, tolmetin, ketorolac, ibuprofen and derivatives, mefenamic acid, meclofenamic acid, piroxicam, tenoxicam, phenylbutazone, oxyphenbutazone, nabumetone, auranofin, aurothioglucose, gold sodium thiomalate, a mixture of any of these, or derivatives of any of these.

Other biologically useful compounds that can also be included in the coating material include, but are not limited to, hormones, (3-blockers, anti-anginal agents, cardiac inotropic agents, corticosteroids, analgesics, anti-inflammatory agents, anti-arrhythmic agents, immunosuppressants, anti-bacterial agents, anti-hypertensive agents, antimalarials, anti-neoplastic agents, anti-protozoal agents, anti-thyroid agents, sedatives, hypnotics and neuroleptics, diuretics, anti-parkinsonian agents, gastro-intestinal agents, anti-viral agents, anti-diabetics, anti-epileptics, anti-fungal agents, histamine H-receptor antagonists, lipid regulating agents, muscle relaxants, nutritional agents such as vitamins and minerals, stimulants, nucleic acids, polypeptides, and vaccines.

Antibiotics are substances which inhibit the growth of or kill microorganisms. Antibiotics can be produced synthetically or by microorganisms. Examples of antibiotics include penicillin, tetracycline, chloramphenicol, minocycline, doxycycline, vancomycin, bacitracin, kanamycin, neomycin, gentamycin, erythromycin, geldanamycin, geldanamycin analogs, cephalosporins, or the like. Examples of cephalosporins include cephalothin, cephapirin, cefazolin, cephalexin, cephradine, cefadroxil, cefamandole, cefoxitin, cefaclor, cefuroxime, cefonicid, ceforanide, cefotaxime, moxalactam, ceftizoxime, ceftriaxone, and cefoperazone.

Antiseptics are recognized as substances that prevent or arrest the growth or action of microorganisms, generally in a nonspecific fashion, e.g., either by inhibiting their activity or destroying them. Examples of antiseptics include silver sulfadiazine, chlorhexidine, glutaraldehyde, peracetic acid, sodium hypochlorite, phenols, phenolic compounds, iodophor compounds, quaternary ammonium compounds, and chlorine compounds.

Antiviral agents are substances capable of destroying or suppressing the replication of viruses. Examples of anti-viral agents include a-methyl-ladamantanemethylamine, hydroxy-ethoxymethylguanine, adamantanamine, 5-iodo-2′-deoxyuridine, trifluorothymidine, interferon, and adenine arabinoside.

Enzyme inhibitors are substances that inhibit an enzymatic reaction. Examples of enzyme inhibitors include edrophonium chloride, N-methylphysostigmine, neostigmine bromide, physostigmine sulfate, tacrine HCL, tacrine, 1-hydroxy maleate, iodotubercidin, p-bromotetramisole, 10-(a-diethylaminopropionyl)-phenothiazine hydrochloride, calmidazolium chloride, hemicholinium-3,3,5-dinitrocatechol, diacylglycerol kinase inhibitor I, diacylglycerol kinase inhibitor II, 3-phenylpropargylaminie, N-monomethyl-L-arginine acetate, carbidopa, 3-hydroxybenzylhydrazine HCl, hydralazine HCl, clorgyline HCl, deprenyl HCl L(−), deprenyl HCl D(+), hydroxylamine HCl, iproniazid phosphate, 6-MeO-tetrahydro-9H-pyrido-indole, nialamide, pargyline HCl, quinacrine HCl, semicarbazide HCl, tranylcypromine HCl, N,N-diethylaminoethyl-2,2-diphenylvalerate hydrochloride, 3-isobutyl-1-methylxanthne, papaverine HCl, indomethacind, 2-cyclooctyl-2-hydroxyethylamine hydrochloride, 2,3-dichloro-a-methylbenzylamine (DCMB), 8,9-dichloro-2,3,4,5-tetrahydro-1H-2-benzazepine hydrochloride, p-aminoglutethimide, p-aminoglutethimide tartrate R(+), paminoglutethimide tartrate S(−), 3-iodotyrosine, alpha-methyltyrosine L(−), alphamethyltyrosine D(−), cetazolamide, dichlorphenamide, 6-hydroxy-2-benzothiazolesulfonamide, and allopurinol.

Anti-pyretics are substances capable of relieving or reducing fever. Anti-inflammatory agents are substances capable of counteracting or suppressing inflammation. Examples of such agents include aspirin (salicylic acid), indomethacin, sodium indomethacin trihydrate, salicylamide, naproxen, colchicine, fenoprofen, sulindac, diflunisal, diclofenac, indoprofen and sodium salicylamide.

Local anesthetics are substances that have an anesthetic effect in a localized region. Examples of such anesthetics include procaine, lidocaine, tetracaine and dibucaine.

Imaging agents are agents capable of imaging a desired site, e.g., tumor, in vivo. Examples of imaging agents include substances having a label that is detectable in vivo, e.g., antibodies attached to fluorescent labels. The term antibody includes whole antibodies or fragments thereof.

Cell response modifiers are chemotactic factors such as platelet-derived growth factor (PDGF). Other chemotactic factors include neutrophil-activating protein, monocyte chemoattractant protein, macrophage-inflammatory protein, SIS (small inducible secreted), platelet factor, platelet basic protein, melanoma growth stimulating activity, epidermal growth factor, transforming growth factor alpha, fibroblast growth factor, platelet-derived endothelial cell growth factor, insulin-like growth factor, nerve growth factor, bone growth/cartilage-inducing factor (alpha and beta), and matrix metalloproteinase inhibitors. Other cell response modifiers are the interleukins, interleukin receptors, interleukin inhibitors, interferons, including alpha, beta, and gamma; hematopoietic factors, including erythropoietin, granulocyte colony stimulating factor, macrophage colony stimulating factor and granulocyte-macrophage colony stimulating factor; tumor necrosis factors, including alpha and beta; transforming growth factors (beta), including beta-1, beta-2, beta-3, inhibin, activin, and DNA that encodes for the production of any of these proteins, antisense molecules, androgenic receptor blockers and statin agents.

In an embodiment, the active agent can be in a microparticle. In an embodiment, microparticles can be dispersed on the surface of the substrate.

The weight of the coating attributable to the active agent can be in any range desired for a given active agent in a given application. In some embodiments, weight of the coating attributable to the active agent is in the range of about 1 microgram to about 10 milligrams of active agent per cm² of the effective surface area of the device. By “effective” surface area it is meant the surface amenable to being coated with the composition itself. For a flat, nonporous, surface, for instance, this will generally be the macroscopic surface area itself, while for considerably more porous or convoluted (e.g., corrugated, pleated, or fibrous) surfaces the effective surface area can be significantly greater than the corresponding macroscopic surface area. In an embodiment, the weight of the coating attributable to the active agent is between about 0.01 mg and about 0.5 mg of active agent per cm² of the gross surface area of the device. In an embodiment, the weight of the coating attributable to the active agent is greater than about 0.01 mg.

In some embodiments, more than one active agent can be used in the coating. Specifically, co-agents or co-drugs can be used. A co-agent or co-drug can act differently than the first agent or drug. The co-agent or co-drug can have an elution profile that is different than the first agent or drug.

In some embodiments, the active agent can be hydrophilic. In an embodiment, the active agent can have a molecular weight of less than 1500 daltons and can have a water solubility of greater than 10 mg/mL at 25° C. In some embodiments, the active agent can be hydrophobic. In an embodiment, the active agent can have a water solubility of less than 10 mg/mL at 25° C.

Some embodiments of the invention include a stent coated with a coating compositon including a first polymer, a second polymer, and at least one bioactive agent selected from the group of steroids and antiproliferatives. In some embodiments, the invention includes a wound dressing coated with a coating composition including a first polymer, a second polymer, and at least one bioactive agent selected from the group consisting of anesthetics, such as procaine, lidocaine, tetracaine and/or dibucaine.

A comprehensive listing of bioactive agents can be found in The Merck Index, Thirteenth Edition, Merck & Co. (2001), the entire contents of which is incorporated by reference herein. Bioactive agents are commercially available from Sigma Aldrich (e.g., vincristine sulfate). The concentration of the bioactive agent or agents dissolved or suspended in the coating mixture can range from about 0.01 to about 90 percent, by weight, based on the weight of the final coated composition. Additives such as inorganic salts, BSA (bovine serum albumin), and inert organic compounds can be used to alter the profile of bioactive agent release, as known to those skilled in the art.

In some embodiments, in order to provide a coating of the present invention, a coating composition is prepared to include one or more solvents, a combination of complementary polymers dissolved in the solvent, and the bioactive agent or agents dispersed in the polymer/solvent mixture. The solvent, in some embodiments, is one in which the polymers form a true solution. The pharmaceutical agent itself may either be soluble in the solvent or form a dispersion throughout the solvent. Suitable solvents include, but are not limited to, alcohols (e.g., methanol, butanol, propanol and isopropanol), alkanes (e.g., halogenated or unhalogenated alkanes such as hexane, cyclohexane, methylene chloride and chloroform), amides (e.g., dimethylformamide), ethers (e.g., tetrahydrofuran (THF), dioxolane, and dioxane), ketones (e.g., methyl ethyl ketone), aromatic compounds (e.g., toluene and xylene), nitriles (e.g., acetonitrile) and esters (e.g., ethyl acetate). In some embodiments, THF and chloroform have been found to be effective solvents due to their excellent solvency for a variety of polymers and bioactive agents of the present invention.

A coating composition of this invention can be used to coat the surface of a variety of devices, and is particularly useful for those devices that will come in contact with aqueous systems. Such devices are coated with a coating composition adapted to release bioactive agent in a prolonged and controlled manner, generally beginning with the initial contact between the device surface and its aqueous environment.

The coated composition provides a means to deliver bioactive agents from a variety of biomaterial surfaces. Various biomaterials include those formed of synthetic polymers, including oligomers, homopolymers, and copolymers resulting from either addition or condensation polymerizations. Examples of suitable addition polymers include, but are not limited to, acrylics such as those polymerized from methyl acrylate, methyl methacrylate, hydroxyethyl methacrylate, hydroxyethyl acrylate, acrylic acid, methacrylic acid, glyceryl acrylate, glyceryl methacrylate, methacrylamide, and acrylamide; vinyls, such as those polymerized from ethylene, propylene, styrene, vinyl chloride, vinyl acetate, vinyl pyrrolidone, and vinylidene difluoride. Examples of condensation polymers include, but are not limited to, nylons such as polycaprolactam, poly(lauryl lactam), poly(hexamethylene adipamide), and poly(hexamethylene dodecanediamide), and also polyurethanes, polycarbonates, polyamides, polysulfones, poly(ethylene terephthalate), poly(lactic acid), poly(glycolic acid), poly(lactic acid-co-glycolic acid), polydimethylsiloxanes, polyetheretherketone, poly(butylene terephthalate), poly(butylene terephthalate-co-polyethylene glycol terephthalate), esters with phosphorus containing linkages, non-peptide polyamino acid polymers, polyiminocarbonates, amino acid-derived polycarbonates and polyarylates, and copolymers of polyethylene oxides with amino acids or peptide sequences.

Certain natural materials are also suitable biomaterials, including human tissue such as bone, cartilage, skin and teeth; and other organic materials such as wood, cellulose, compressed carbon, and rubber. Other suitable biomaterials include metals and ceramics. The metals include, but are not limited to, titanium, stainless steel, and cobalt chromium. A second class of metals include the noble metals such as gold, silver, copper, and platinum. Alloys of metals, such as nitinol (e.g. MP35), may be suitable for biomaterials as well. The ceramics include, but are not limited to, silicon nitride, silicon carbide, zirconia, and alumina, as well as glass, silica, and sapphire. Yet other suitable biomaterials include combinations of ceramics and metals, as well as biomaterials that are fibrous or porous in nature.

Optionally, the surface of some biomaterials can be pretreated (e.g., with a silane and/or Parylene™ coating composition in one or more layers) in order to alter the surface properties of the biomaterial. For example, in various embodiments of the present invention a layer of silane may be applied to the surface of the biomaterial followed by a layer of Parlene™. Parylene™ C is the polymeric form of the low-molecular-weight dimer of para-chloro-xylylene. Silane and/or Parylene™ C (a material supplied by Specialty Coating Systems (Indianapolis)) can be deposited as a continuous coating on a variety of medical device parts to provide an evenly distributed, transparent layer. In one embodiment, the deposition of Parylene™ is accomplished by a process termed vapor deposition polymerization, in which dimeric Parylene™ C is vaporized under vacuum at 150° C., pyrolyzed at 680° C. to form a reactive monomer, then pumped into a chamber containing the component to be coated at 25° C. At the low chamber temperature, the monomeric xylylene is deposited on the part, where it immediately polymerizes via a free-radical process. The polymer coating reaches molecular weights of approximately 500 kilodaltons.

Deposition of the xylylene monomer takes place in only a moderate vacuum (0.1 torr) and is not line-of-sight. That is, the monomer has the opportunity to surround all sides of the part to be coated, penetrating into crevices or tubes and coating sharp points and edges, creating what is called a “conformal” coating. With proper process control, it is possible to deposit a pinhole-free, insulating coating that will provide very low moisture permeability and high part protection to corrosive biological fluids.

Adherence is a function of the chemical nature of the surface to be coated. It has been reported, for instance, that tantalum and silicon surfaces can be overcoated with silicon dioxide, then with plasma-polymerized methane, and finally with Parylene™ C to achieve satisfactory adherence.

Most applications of Parylene™ C coating in the medical device industry are for protecting sensitive components from corrosive body fluids or for providing lubricity to surfaces. Typical anticorrosion applications include blood pressure sensors, cardiac-assist devices, prosthetic components, bone pins, electronic circuits, ultrasonic transducers, bone-growth stimulators, and brain probes. Applications to promote lubricity include mandrels, injection needles, cannulae, and catheters.

Also, as previously described above, the surface to which the composition is applied can itself be pretreated in other manners sufficient to improve attachment of the composition to the underlying (e.g., metallic) surface. Additional examples of such pretreatments include photografted polymers, epoxy primers, polycarboxylate resins, and physical roughening of the surface. It is further noted that the pretreatment compositions and/or techniques may be used in combination with each other or may be applied in separate layers to form a pretreatment coating on the surface of the medical device.

As described above, the surface of a medical device may be roughened to increase adhesion of the coating composition to the medical device and/or alter elution profiles. Without intending to be bound by theory, the roughening of the surface provides for a greater surface area between the coating composition and the surface of the medical device, which may increase adhesion. Further, in embodiments with relatively aggressive roughening and/or relatively thin coatings, the peaks and valleys of the roughened surface may transfer through the coating composition, thereby increasing the surface area of the coating. Such increased surface area may alter the bioactive agent release profile in situ.

The surface of the medical device may be roughened by any suitable method. In some embodiments, the surface of the medical device may be roughened by projecting silica particles at the surface. The extent of the roughening may be characterized by peak to valley distances. For example, the extent of roughening may be characterized by the distance between the average of the ten highest peaks and the ten lowest valleys. In some embodiments, the extent of roughening may range from about 2 μm to about 20 μm. Optionally, the extent of roughening may range from about 5 μm to about 15 μm. In some embodiments, the extent of roughening may range from about 6.5 μm to about 12 μm.

In some embodiments, a tie-in layer may be utilized to facilitate one or more physical and/or covalent bonds between layers. For example, the pretreatment layer may include a multi-interface system to facilitate adhesion and cohesion interaction relative to the different materials positioned at the interface of each layer. For example, the application of Parylene pretreatments to metal surfaces may be aided by a first application of a reactive organosilane reagent. A reactive organosilane reagent containing an unsaturated pendant group is capable of participating with the Parylene radicals as they deposit on the surface from the vapor phase. After cleaning of the metal surface, an organosilane reagent with an unsaturated pendant group may be applied to the metal oxide surface on a metal substrate. Without intending to be bound by theory, it appears that the silicon in the organosilane reagent couples covalently to the metal oxide, linking the organosilane group to the surface. The substrate may then be placed in a Parylene reactor and exposed to the vapor-phase Parylene process. During this process, the unsaturated pendant groups on the organosilane-treated surface can react with the Parylene diradicals depositing from the vapor phase. This forms a covalent link between the Parylene and the organosilane layer. The Parylene also forms covalent bonds to itself as it deposits. Thus, this process yields a layered surface in which the layers are covalently bonded to each other. This forms a very strong bond between the Parylene and the metal surface, resulting in high durability to mechanical challenges. Further, in some embodiments, the Parylene may physically bond with the bioactive agent delivery coating or may include a reactive acrylate group that can be reacted with the bioactive agent delivery coating to improve durability to mechanical challenges.

The coating composition of the present invention can be used in combination with a variety of devices, including those used on a temporary, transient, or permanent basis upon and/or within the body.

Compositions of this invention can be used to coat the surface of a variety of implantable devices, for example: drug-delivering vascular stents (e.g., self-expanding stents typically made from nitinol, balloon-expanded stents typically prepared from stainless steel); other vascular devices (e.g., grafts, catheters, valves, artificial hearts, heart assist devices); implantable defibrillators; blood oxygenator devices (e.g., tubing, membranes); surgical devices (e.g., sutures, staples, anastomosis devices, vertebral disks, bone pins, suture anchors, hemostatic barriers, clamps, screws, plates, clips, vascular implants, tissue adhesives and sealants, tissue scaffolds); membranes; cell culture devices; chromatographic support materials; biosensors; shunts for hydrocephalus; wound management devices; endoscopic devices; infection control devices; orthopedic devices (e.g., for joint implants, fracture repairs); dental devices (e.g., dental implants, fracture repair devices), urological devices (e.g., penile, sphincter, urethral, bladder and renal devices, and catheters); colostomy bag attachment devices; ophthalmic devices (e.g. ocular coils); glaucoma drain shunts; synthetic prostheses (e.g., breast); intraocular lenses; respiratory, peripheral cardiovascular, spinal, neurological, dental, ear/nose/throat (e.g., ear drainage tubes); renal devices; and dialysis (e.g., tubing, membranes, grafts).

Examples of useful devices include urinary catheters (e.g., surface-coated with antimicrobial agents such as vancomycin or norfloxacin), intravenous catheters (e.g., treated with antithrombotic agents (e.g., heparin, hirudin, coumadin), small diameter grafts, vascular grafts, artificial lung catheters, atrial septal defect closures, electro-stimulation leads for cardiac rhythm management (e.g., pacer leads), glucose sensors (long-term and short-term), degradable coronary stents (e.g., degradable, non-degradable, peripheral), blood pressure and stent graft catheters, birth control devices, benign prostate and prostate cancer implants, bone repair/augmentation devices, breast implants, cartilage repair devices, dental implants, implanted drug infusion tubes, intravitreal drug delivery devices, nerve regeneration conduits, oncological implants, electrostimulation leads, pain management implants, spinal/orthopedic repair devices, wound dressings, embolic protection filters, abdominal aortic aneurysm grafts, heart valves (e.g., mechanical, polymeric, tissue, percutaneous, carbon, sewing cuff), valve annuloplasty devices, mitral valve repair devices, vascular intervention devices, left ventricle assist devices, neuro aneurysm treatment coils, neurological catheters, left atrial appendage filters, hemodialysis devices, catheter cuff, anastomotic closures, vascular access catheters, cardiac sensors, uterine bleeding patches, urological catheters/stents/implants, in vitro diagnostics, aneurysm exclusion devices, and neuropatches.

Examples of other suitable devices include, but are not limited to, vena cava filters, urinary dialators, endoscopic surgical tissue extractors, atherectomy catheters, clot extraction catheters, percutaneous transluminal angioplasty catheters, PTCA catheters, stylets (vascular and non-vascular), coronary guidewires, drug infusion catheters, esophageal stents, circulatory support systems, angiographic catheters, transition sheaths and dilators, coronary and peripheral guidewires, hemodialysis catheters, neurovascular balloon catheters, tympanostomy vent tubes, cerebro-spinal fluid shunts, defibrillator leads, percutaneous closure devices, drainage tubes, thoracic cavity suction drainage catheters, electrophysiology catheters, stroke therapy catheters, abscess drainage catheters, biliary drainage products, dialysis catheters, central venous access catheters, and parental feeding catheters.

Examples of medical devices suitable for the present invention include, but are not limited to catheters, implantable vascular access ports, blood storage bags, vascular stents, blood tubing, arterial catheters, vascular grafts, intraaortic balloon pumps, cardiovascular sutures, total artificial hearts and ventricular assist pumps, extracorporeal devices such as blood oxygenators, blood filters, hemodialysis units, hemoperfusion units, plasmapheresis units, hybrid artificial organs such as pancreas or liver and artificial lungs, as well as filters adapted for deployment in a blood vessel in order to trap emboli (also known as “distal protection devices”).

The compositions are particularly useful for those devices that will come in contact with aqueous systems, such as bodily fluids. Such devices are coated with a coating composition adapted to release bioactive agent in a prolonged and controlled manner, generally beginning with the initial contact between the device surface and its aqueous environment. It is important to note that the local delivery of combinations of bioactive agents may be utilized to treat a wide variety of conditions utilizing any number of medical devices, or to enhance the function and/or life of the device. Essentially, any type of medical device may be coated in some fashion with one or more bioactive agents that enhances treatment over use of the individual use of the device or bioactive agent.

In various embodiments, the coating composition can also be used to coat stents, e.g., either self-expanding stents, which are typically prepared from nitinol, or balloon-expandable stents, which are typically prepared from stainless steel. Other stent materials, such as cobalt chromium alloys, can be coated by the coating composition as well.

Devices which are particularly suitable include vascular stents such as self-expanding stents and balloon expandable stents. Examples of self-expanding stents useful in the present invention are illustrated in U.S. Pat. Nos. 4,655,771 and 4,954,126 issued to Wallsten and U.S. Pat. No. 5,061,275 issued to Wallsten et al. Examples of suitable balloon-expandable stents are shown in U.S. Pat. No. 4,733,665 issued to Palmaz, U.S. Pat. No. 4,800,882 issued to Gianturco and U.S. Pat. No. 4,886,062 issued to Wiktor.

In other embodiments, the coating composition can also be used to coat ophthalmic devices, e.g. ocular coils. A therapeutic agent delivery device that is particularly suitable for delivery of a therapeutic agent to limited access regions, such as the vitreous chamber of the eye and inner ear is described in U.S. Pat. No. 6,719,750 and U.S. Patent Application Publication No. 2005/0019371 A1.

The resultant coating composition can be applied to the device in any suitable fashion (e.g., the coating composition can be applied directly to the surface of the medical device, or alternatively, to the surface of a surface-modified medical device, by dipping, spraying, ultrasonic deposition, or using any other conventional technique). The suitability of the coating composition for use on a particular material, and in turn, the suitability of the coated composition can be evaluated by those skilled in the art, given the present description. In one such embodiment, for instance, the coating comprises at least two layers which are themselves different. For instance, a base layer may be applied having bioactive agent(s) alone, or together with or without one or more of the polymer components, after which one or more topcoat layers are coated, each with either first and/or second polymers as described herein, and with or without bioactive agent. These different layers, in turn, can cooperate in the resultant composite coating to provide an overall release profile having certain desired characteristics, and in some embodiments, for use with bioactive agents of high molecular weight. In some embodiments, the composition is coated onto the device surface in one or more applications of a single composition that includes first and second polymers, together with bioactive agent. However, as previously suggested a pretreatment layer or layers may be first applied to the surface of the device, wherein subsequent coating with the composition may be performed onto the pretreatment layer(s). The method of applying the coating composition to the device is typically governed by the geometry of the device and other process considerations. The coating is subsequently cured by evaporation of the solvent. The curing process can be performed at room or elevated temperature, and optionally with the assistance of vacuum and/or controlled humidity.

It is also noted that one or more additional layers may be applied to the coating layer(s) that include bioactive agent. Such layer(s) or topcoats can be utilized to provide a number of benefits, such as biocompatibility enhancement, delamination protection, durability enhancement, bioactive agent release control, to just mention a few. In one embodiment the topcoat may include one or more of the first, second, and/or additional polymers described herein without the inclusion of a bioactive agent. In various embodiments, the topcoat includes a second polymer that is a poly(alkyl(meth)acrylate). An example of one embodiment of a poly(alkyl(meth)acrylate) includes poly(n-butyl methacrylate). In another embodiment, the first or second polymers could further include functional groups (e.g. hydroxy, thiol, methylol, amino, and amine-reactive functional groups such as isocyanates, thioisocyanates, carboxylic acids, acyl halides, epoxides, aldehydes, alkyl halides, and sulfonate esters such as mesylate, tosylate, and tresylate) that could be utilized to bind the topcoat to the adjacent coating composition. In another embodiment of the present invention one or more of the pretreatment materials (e.g. Parylene™) may be applied as a topcoat. Additionally, biocompatible topcoats (e.g. heparin, collagen, extracellular matrices, cell receptors . . . ) may be applied to the coating composition of the present invention. Such biocompatible topcoats may be adjoined to the coating composition of the present invention by utilizing photochemical or thermochemical techniques known in the art. Additionally, release layers may be applied to the coating composition of the present invention as a friction barrier layer or a layer to protect against delamination. Examples of biocompatible topcoats that may be used include those disclosed in U.S. Pat. No. U.S. Pat. Nos. 4,979,959 and 5,744,515.

Optionally, a hydrophilic topcoat may be provided. Such topcoats may provide several advantages, including providing a relatively more lubricious surface to aid in medical device placement in situ, as well as to further increase biocompatibility in some applications. Examples of hydrophilic agents that may be suitable for a topcoat in accordance with the invention includes polyacrylamide(36%)co-methacrylic acid(MA)-(10%)co-methoxy PEG1000MA-(4%)co-BBA-APMA compounds such as those described in example 4 of US Patent Application Publication No. 2002/0041899, photoheparin such as described in example 4 of U.S. Pat. No. 5,563,056, and a photoderivatized coating as described in Example 1 of U.S. Pat. No. 6,706,408, the contents of each of which is hereby incorporated by reference.

In some embodiments, the topcoat may be used to control the elution rate of a bioactive agent from a medical device surface. For example, topcoats may be described as the weight of the topcoat relative to the weight of the underlying bioactive agent containing layer. For example, the topcoat may be about 1 percent to about 50 percent by weight relative to the underlying layer. In some embodiments, the topcoat may be about 2 percent to about 25 percent by weight relative to the underlying layer. Optionally, in some embodiments, the topcoat may be about 5 percent to about 12 percent by weight relative to the underlying layer.

Applicants have found that providing a relatively thin topcoat compared to the underlying layer may significantly reduce initial drug elution rates to provide for longer elution times. For example, providing a topcoat weighing about 5% of the underlying layer may reduce initial elution rates (e.g., less than 20 hours) by more than about 50%.

In some embodiments, the topcoat layer comprises a polymer that is also included in the underlying layer (e.g., first, second, and/or additional polymers as described above). Such topcoats may provide for superior adhesion between the top coat and the underlying layer.

Further, in some embodiments, one or more bioactive agents may be provided in a topcoat (sometimes referred to herein as a topcoat bioactive agent). The topcoat bioactive agent may be the same as or distinguishable from the bioactive agent included in an underlying layer. Providing bioactive agent within the topcoat allows for the bioactive agent to be in contact with surrounding tissue in situ while providing a longer release profile compared to coating compositions provided without topcoats. Such topcoats may also be used to further control the elution rate of a bioactive agent from a medical device surface, such as by varying the amount of bioactive agent in the topcoat. The degree to which the bioactive agent containing topcoat affects elution will depend on the specific bioactive agent within the topcoat as well as the concentration of the bioactive agent within the topcoat.

Any suitable amount of a bioactive agent may be included in the topcoat. For example, the upper limit of the amount of bioactive agent in the topcoat may be limited only by the ability of the topcoat to hold additional bioactive agent. In some embodiments, the bioactive agent may comprise about 1 to about 75 percent of the topcoat. Optionally, the bioactive agent may comprise about 5 to about 50 percent of the topcoat. In yet other embodiments, the bioactive agent may comprise about 10 to about 40 percent of the topcoat.

The polymer composition for use in this invention is generally biocompatible, e.g., such that it results in no significant induction of inflammation or irritation when implanted. In addition, the polymer combination is generally useful throughout a broad spectrum of both absolute concentrations and relative concentrations of the polymers. This means that the physical characteristics of the coating, such as tenacity, durability, flexibility and expandability, will typically be adequate over a broad range of polymer concentrations. In turn, the ability of the coating to control the release rates of a variety of bioactive agents can be manipulated by varying the absolute and relative concentrations of the polymers.

Additionally, the coatings of the present invention are generally hydrophobic and limit the intake of aqueous fluids. For example, many embodiments of the present invention are coating compositions including two or more hydrophobic polymers wherein the resulting coating shows <10% (wt) weight change when exposed to water, and in some embodiments <5% (wt) weight change when exposed to water.

A coating composition can be provided in any suitable form, e.g., in the form of a true solution, or fluid or paste-like emulsion, mixture, dispersion or blend. In some embodiments, polymer combinations of this invention are capable of being provided in the form of a true solution, and in turn, can be used to provide a coating that is both optically clear (upon microscopic examination), while also containing a significant amount of bioactive agent. In turn, the coated composition will generally result from the removal of solvents or other volatile components and/or other physical-chemical actions (e.g., heating or illuminating) affecting the coated composition in situ upon the surface.

A further example of a coating composition embodiment may include a configuration of one or more bioactive agents within an inner matrix structure, for example, bioactive agents within or delivered from a degradable encapsulating matrix or a microparticle structure formed of semipermeable cells and/or degradable polymers. One or more inner matrices may be placed in one or more locations within the coating composition and at one or more locations in relation to the substrate. Examples of inner matrices, for example degradable encapsulating matrices formed of semipermeable cells and/or degradable polymers, are disclosed and/or suggested in U.S. Publication No. 20030129130, U.S. Patent Application Ser. No. 60/570,334 filed May 12, 2004, U.S. Patent Application Ser. No. 60/603,707, filed Aug. 23, 2004, U.S. Publication No. 20040203075, filed Apr. 10, 2003, U.S. Publication No. 20040202774 filed on Apr. 10, 2003, and U.S. patent application Ser. No. 10/723,505, filed Nov. 26, 2003, the entire contents of which are incorporated by reference herein.

The overall weight of the coating upon the surface may vary depending on the application. However, in some embodiments, the weight of the coating attributable to the bioactive agent is in the range of about one microgram to about 10 milligram (mg) of bioactive agent per cm² of the effective surface area of the device. By “effective” surface area it is meant the surface amenable to being coated with the composition itself. For a flat, nonporous, surface, for instance, this will generally be the macroscopic surface area itself, while for considerably more porous or convoluted (e.g., corrugated, pleated, or fibrous) surfaces the effective surface area can be significantly greater than the corresponding macroscopic surface area. In various embodiments, the weight of the coating attributable to the bioactive agent is between about 0.005 mg and about 10 mg, and in some embodiments between about 0.01 mg and about 1 mg of bioactive agent per cm² of the gross surface area of the device. This quantity of bioactive agent is generally required to provide desired activity under physiological conditions.

In turn, in various embodiments, the final coating thickness of a coated composition will typically be in the range of about 0.1 micrometers to about 100 micrometers, and in some embodiments, between about 0.5 micrometers and about 25 micrometers. This level of coating thickness is generally required to provide an adequate concentration of drug to provide adequate activity under physiological conditions.

The invention will be further described with reference to the following non-limiting Examples. It will be apparent to those skilled in the art that many changes can be made in the embodiments described without departing from the scope of the present invention. Thus the scope of the present invention should not be limited to the embodiments described in this application, but only by the embodiments described by the language of the claims and the equivalents of those embodiments. Unless otherwise indicated, all percentages are by weight.

### EXAMPLES

### Test Procedures

The potential suitability of particular coated compositions for in vivo use can be determined by a variety of screening methods, examples of each of which are described herein. Not all of these test procedures were used in connection with the example included in this application, but they are described here to enable consistent comparison of coatings in accordance with the invention.

Sample Preparation Procedure

Stainless steel stents used in the following examples were manufactured by Laserage Technology Corporation, Waukegan, Ill. In some cases, the metal surface of the stents may be coated without any pretreatment beyond washing. In other cases, a primer may be applied to the stents by first cleaning the stents with aqueous base, then pre-treating with a silane followed by vapor deposition of Parylene™ polymer. The silane used may be [3-(methacroyloxy)propyl]trimethoxysilane, available from Sigma-Aldrich Fine Chemicals as Product No. 44,015-9. The silane may be applied as essentially a monolayer by mixing the silane at a low concentration in 50/50 (vol) isopropanol/water, soaking the stents in the aqueous silane solution for a suitable length of time to allow the water to hydrolyze the silane and produce some cross-linking, washing off residual silane, then baking the silane-treated stent at 100° C. for conventional periods of time. Following the silane treatment, Parylene™ C coating (available from Union Carbide Corporation, Danbury, Conn.) may be vapor-deposited at a thickness of about 1 mm. Prior to coating, the stents should be weighed on a microbalance to determine a tare weight.

Bioactive agent/polymer solutions may be prepared at a range of concentrations in an appropriate solvent (typically tetrahydrofuran or chloroform), in the manner described herein. In all cases the coating solutions are applied to respective stents by spraying, and the solvent is allowed to evaporate under ambient conditions. The coated stents are then re-weighed to determine the mass of coating and consequently the mass of polymer and bioactive agent.

Rapamycin Release Assay Procedure

The Rapamycin Release Assay Procedure, as described herein, was used to determine the extent and rate of release of an exemplary bioactive agent, rapamycin, under in vitro elution conditions. Spray-coated stents prepared using the Sample Preparation Procedure were placed in sample baskets into 10 milliliters of Sotax™ dissolution system (elution media containing 2% (wt) surfactant/water solution, available from Sotax Corporation, Horsham, Pa.). Amount of bioactive agent elution was monitored by UV spectrometry over the course of several days. The elution media was held at 37° C. After the elution measurements, the stents were removed, rinsed, dried, and weighed to compare measured bioactive agent elution to weighed mass loss.

Dexamethasone Release Assay Procedure

The Dexamethasone Release Assay Procedure, as described herein, may be used to determine the extent and rate of dexamethasone release under in vitro conditions. Spray-coated stents made using the Sample Preparation Procedure are placed in 10 milliliters of pH 7 phosphate buffer solution (“PBS”) contained in an amber vial. A magnetic stirrer bar is added to the vial, and the vial with its contents are placed into a 37° C. water bath. After a sample interval, the stent is removed and placed into a new buffer solution contained in a new vial. Dexamethasone concentration in the buffer is measured using ultraviolet spectroscopy and the concentration converted to mass of bioactive agent released from the coating. After the experiment, the stent is dried and weighed to correlate actual mass loss to the loss measured by the elution experiment.

Durability Test Procedure

The durability of the coated composition can be determined by the following manner. To simulate use of the coated devices, the coated stents are placed over sample angioplasty balloons. The stent is then crimped onto the balloon using a laboratory test crimper (available from Machine Solutions, Brooklyn, N.Y.). The stent and balloon are then placed in a phosphate buffer bath having a pH of 7.4 and temperature of 37° C. After 5 minutes of soaking, the balloon is expanded using air at 5 atmospheres (3800 torr) of pressure. The balloon is then deflated, and the stent is removed.

The stent is then examined by optical and scanning electron microscopy to determine the amount of coating damage caused by cracking and/or delamination and a rating may be assigned. Coatings with extensive damage are considered unacceptable for a commercial medical device. The “Rating” is a qualitatitive scale used to describe the amount of damage to the coating from the stent crimping and expansion procedure based on optical microscopy examination by an experienced coating engineer. A low rating indicates a large percentage of the coating cracked, smeared, and/or delaminated from the surface. For example, a coating with a rating of ten shows no damage while one with a rating of 1 will show a majority of the coating damaged to the point where clinical efficacy maybe diminished. Commercially attractive coatings typically have a rating of nine or higher.

Stress-Strain Measurement Test Procedure

Polymer films can be prepared by hot pressing polymer beads at 100° C. in a constant film maker kit to a thickness of approximately 0.5 mm. The resulting films are cut into strips using a razor blade. A Q800 Dynamic Mechanical Analyzer (available from Texas Instruments, Dallas, Tex.) may be fitted with a film tension clamp. Each sample is equilibrated at 35° C. for five minutes prior to straining the sample. Then the sample is loaded into the clamp such that the sample length is between 5 and 7 mm in length. A static force of 0.01N is applied to each sample throughout the measurements. Simultaneously, a 0.5 N/min force is applied to the sample until the movable clamp reaches its maximum position. Films are elongated at constant stress and the average tensile modulus (i.e., the initial slope of the stress-strain curve, in MPa) can be determined.

### Example 1

### Release of Rapamycin from Poly(ethylene-co-propylene) and Poly(butyl methacrylate)

Three solutions were prepared for coating the stents. The solutions included mixtures of poly(ethylene-co-propylene) (“PEPP”, available from Sigma-Aldrich Fine Chemicals, Milwaukee, Wis., as Product No. 18,962-6, contains 60% (mole) ethylene, having M_{w} of approximately 170 kilodaltons ), “PBMA” and “RAPA” (“PBMA”, available from Sigma-Aldrich Fine Chemicals as Product No. 18,152-8, having a weight average molecular weight (Mw) of about 337 kilodaltons), and rapamycin (“RAPA”, available from LC Laboratories, Woburn, Mass.) dissolved in THF to form a homogeneous solution. The stents were not given a primer pre-treatment.

The solutions were prepared to include the following ingredients at the stated weights per milliliter of THF:
1) 16 mg/ml PEPP/4 mg/ml PBMA/10 mg/ml RAPA
2) 10 mg/ml PEPP/10 mg/ml PBMA/10 mg/ml RAPA
3) 4 mg/ml PEPP/16 mg/ml PBMA/10 mg/ml RAPA

Using the Sample Preparation Procedure, two stents were spray coated using each solution. After solvent removal via ambient evaporation, the drug elution for each coated stent was monitored using the Rapamycin Release Assay Procedure.

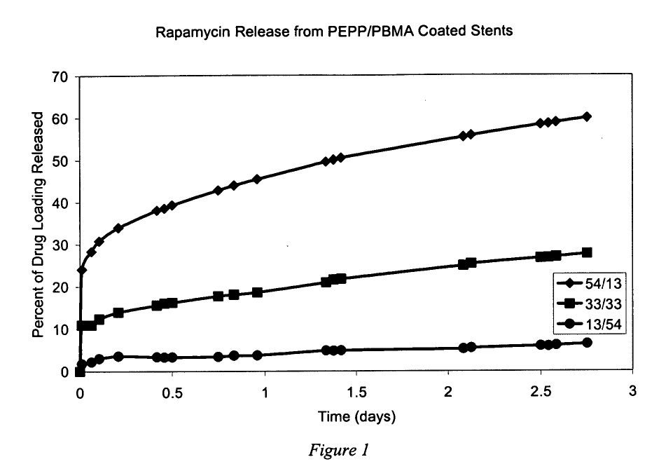

Results, provided in FIG. 1, demonstrate the ability to control the elution rate of rapamycin, a pharmaceutical agent, from a coated stent surface by varying the relative concentrations of PEPP and PBMA in the polymer mixture as described herein.

### Example 2

### Release of Rapamycin from Poly(epichlorohydrin) and Poly(butyl methacrylate)

Three solutions were prepared for coating the stents. The solutions included mixtures of poly(epichlorohydrin) (“PECH”, available from Scientific Polymer Products as Catalog #127, CAS #24969-06-0, M_{w} approximately 700 kilodaltons), poly(butyl methacrylate) (“PBMA”, available from Sigma-Aldrich Fine Chemicals as Product No. 18,152-8, having a weight average molecular weight (Mw) of about 337 kilodaltons), and rapamycin (“RAPA”, available from LC Laboratories, Woburn, Mass.) dissolved in tetrahydrofuran (THF) to form a homogeneous solution. The stents were not given a primer pre-treatment.

The solutions were prepared to include the following ingredients at the stated weights per milliliter of THF:
1) 16 mg/ml PECH/4 mg/ml PBMA/10 mg/ml RAPA
2) 10 mg/ml PECH/10 mg/ml PBMA/10 mg/ml RAPA
3) 4 mg/ml PECH/16 mg/ml PBMA/10 mg/ml RAPA

Using the Sample Preparation Procedure, two stents were spray coated using each solution. After solvent removal via ambient evaporation, the drug elution for each coated stent was monitored using the Rapamycin Release Assay Procedure.

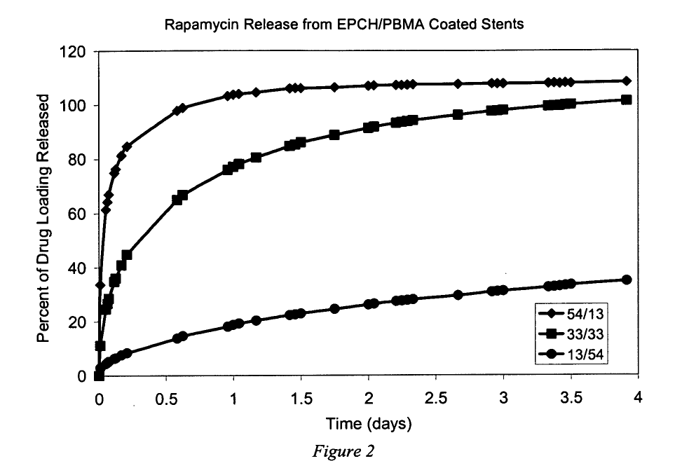

Results, provided in FIG. 2, demonstrate the ability to control the elution rate of rapamycin, a pharmaceutical agent, from a coated stent surface by varying the relative concentrations of PECH and PBMA in the polymer mixture as described herein.

### Example 3

### Release of Rapamycin from Poly(isobutylene) and Poly(butyl methacrylate)

Three solutions were prepared for coating the stents. The solutions included mixtures of poly(isobutylene) (“PIB”, available from Scientific Polymer Products as Catalog #681, CAS #9003-27-4, M_{w} approx. 85 kilodaltons), (“PBMA”, available from Sigma-Aldrich Fine Chemicals as Product No. 18,152-8, having a weight average molecular weight (Mw) of about 337 kilodaltons), and rapamycin (“RAPA”, available from LC Laboratories, Woburn, Mass.) dissolved in THF to form a homogeneous solution. The stents were not given a primer pre-treatment.

The solutions were prepared to include the following ingredients at the stated weights per milliliter of THF:
1) 16 mg/ml PIB/4 mg/ml PBMA/10 mg/ml RAPA
2) 10 mg/ml PIB/10 mg/ml PBMA/10 mg/ml RAPA
3) 4 mg/ml PIB/16 mg/ml PBMA/10 mg/ml RAPA

Using the Sample Preparation Procedure, two stents were spray coated using each solution. After solvent removal via ambient evaporation, the drug elution for each coated stent was monitored using the Rapamycin Release Assay Procedure.

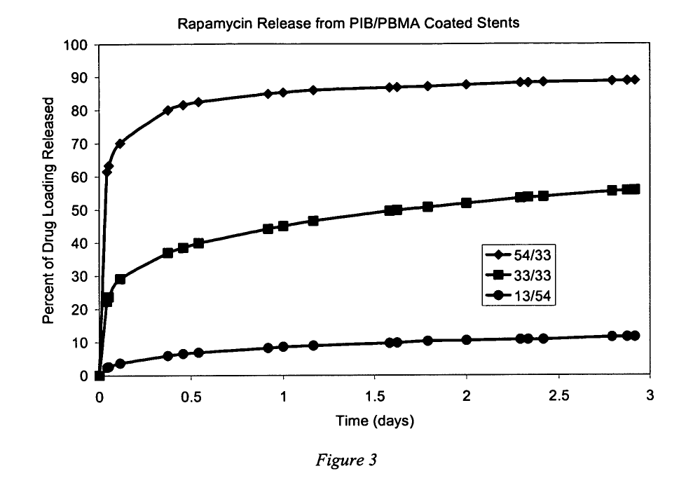

Results, provided in FIG. 3, demonstrate the ability to control the elution rate of rapamycin, a pharmaceutical agent, from a coated stent surface by varying the relative concentrations of P1B and PBMA in the polymer mixture as described herein.

### Example 4

### Release of Rapamycin from Poly(styrene-co-butadiene) and Poly(butyl methacrylate)

Three solutions were prepared for coating the stents. The solutions included mixtures of poly(styrene-co-butadiene) copolymer (“SBR”, available from Scientific Polymer Products, Inc. Catalog #100, contains 23% (wt) styrene), poly(butyl methacrylate) (“PBMA”, available from Sigma-Aldrich Fine Chemicals as Product No. 18,152-8, having a weight average molecular weight (Mw) of about 337 kilodaltons), and rapamycin (“RAPA”, available from LC Laboratories, Woburn, Mass.) dissolved in THF to form a homogeneous solution. The stents were not given a primer pre-treatment.

The solutions were prepared to include the following ingredients at the stated weights per milliliter of THF:
1) 16 mg/ml SBR/4 mg/ml PBMA/10 mg/ml RAPA
2) 10 mg/ml SBR/10 mg/ml PBMA/10 mg/ml RAPA
3) 4 mg/ml SBR/16 mg/ml PBMA/10 mg/ml RAPA

Using the Sample Preparation Procedure, two stents were spray coated using each solution. After solvent removal via ambient evaporation, the drug elution for each coated stent was monitored using the Rapamycin Release Assay Procedure.

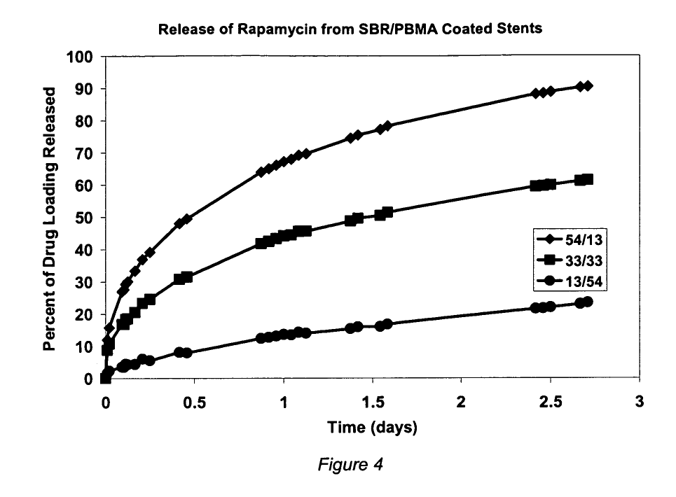

Results, provided in FIG. 4, demonstrate the ability to control the elution rate of rapamycin, a pharmaceutical agent, from a coated stent surface by varying the relative concentrations of SBR and PBMA in the polymer mixture as described herein.

### Example 5

### Release of Rapamycin from Poly(ethylene-co-methyl acrylate) and Poly(butyl methacrylate)

Three solutions were prepared for coating the stents. All three solutions included mixtures of poly(ethylene-co-methyl acrylate) (“PEMA”, available from Focus Chemical Corp. Portsmouth, N.H., containing 28% (wt) methyl acrylate), poly(butyl methacrylate) (“PBMA”, available from Sigma-Aldrich Fine Chemicals as Product No. 18,152-8, having a weight average molecular weight (Mw) of about 337 kilodaltons), and rapamycin (“RAPA”, available from LC Laboratories, Woburn, Mass.) dissolved in tetrahydrofuran (THF) to form a homogeneous solution. The stents were not given a primer pre-treatment.

The solutions were prepared to include the following ingredients at the stated weights per milliliter of THF:
1) 16 mg/ml PEMA/4 mg/ml PBMA/10 mg/ml RAPA
2) 10 mg/ml PEMA/10 mg/ml PBMA/10 mg/ml RAPA
3) 4 mg/ml PEMA/16 mg/ml PBMA/10 mg/ml RAPA

Using the Sample Preparation Procedure, two stents were spray coated using each solution. After solvent removal via ambient evaporation, the drug elution for each coated stent was monitored using the Rapamycin Release Assay Procedure.

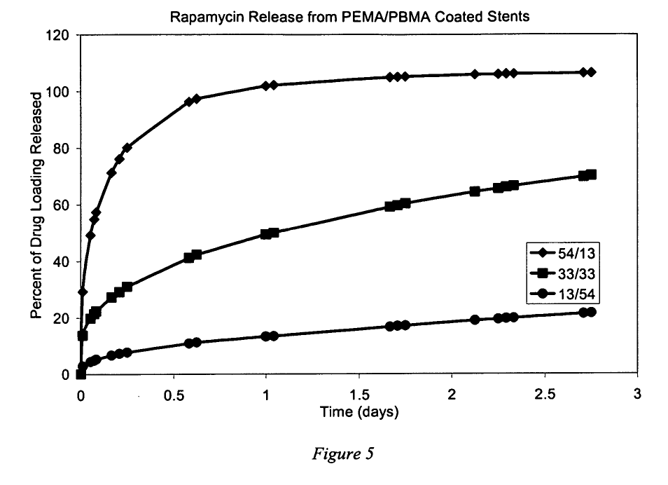

Results, provided in FIG. 5, demonstrate the ability to control the elution rate of rapamycin, a pharmaceutical agent, from a coated stent surface by varying the relative concentrations of PEMA and PBMA in the polymer mixture as described herein. The lines in FIG. 5 and similar figures are expressed in terms of percent by weight of the first and second polymers, respectively, in the coated compositions. This can be compared to the amounts provided above, which are stated in terms of “mg/ml” of the respective polymers in the coating compositions themselves, which are applied to the stents. Hence “54/13” corresponds to the coated compositions that results from the use of the first coating composition above, which upon removal of the solvent provides a coated composition having 54% PEMA and 13% PBMA respectively, by weight. Alternatively, solutions such as the second solution above, e.g., which includes equal amounts (by weight) of the ingredients, will alternatively be referred to herein as “33/33/33”, representing the weight ratio of ingredients to each other.

### Example 6

### Release of Dexamethasone from Poly(ethylene-co-methyl acrylate) and Poly(butyl methacrylate)

Three solutions were prepared for coating the stents. All three solutions included mixtures of poly(ethylene-co-methyl acrylate) (“PEMA”), poly(butyl methacrylate) “PBMA”, and dexamethasone (“DEXA”, available as Product No. 86,187-1 from Sigma Aldrich Fine Chemicals) dissolved in THF to form a homogeneous solution. The stents were not given a primer pre-treatment. The solutions were prepared to include the following ingredients at the stated weights per milliliter of THF:
1) 20 mg/ml PEMA/0 mg/ml PBMA/10 mg/ml DEXA
2) 10 mg/ml PEMA/10 mg/ml PBMA/10 mg/ml DEXA
3) 0 mg/ml PEMA/20 mg/ml PBMA/10 mg/ml DEXA

Using the Sample Preparation Procedure, two stents were spray coated using each solution. After solvent removal via ambient evaporation, the drug elution for each coated stent was monitored using the Dexamethasone Release Assay Procedure.

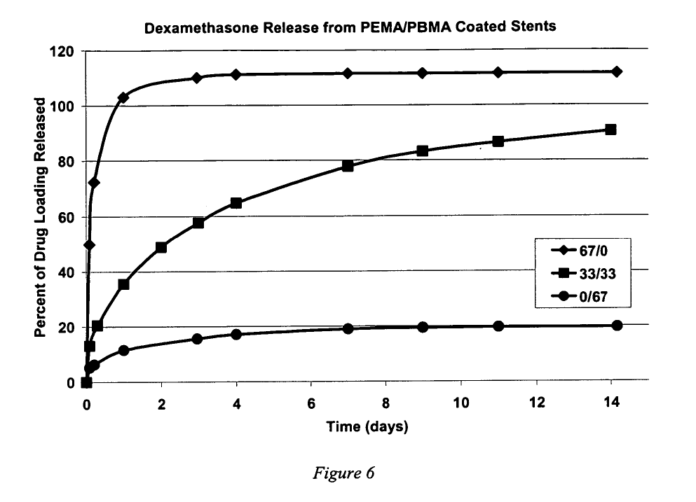

Results, provided in FIG. 6, demonstrate the ability to control the elution rate of dexamethasone, a pharmaceutical agent, from a stent surface by varying the relative concentrations of PEMA and PBMA in the polymer mixture.

### Example 7

### Surface Characterization of Coated Stents after Crimping and Expansion

Using the Sample Preparation Procedure, stents were sprayed with a coating of second polymer/poly(butyl methacrylate)(“PBMA”)/rapamycin(“RAPA”), mixed at a weight ratio of 33/33/33 at 10 mg/ml each of THF. The first polymer was poly(ethylene-co-methyl acrylate) (“PEMA”, available from Focus Chemical Corp. Portsmouth, N.H., containing 28% (wt) methyl acrylate). The second polymer used was PBMA from Sigma-Aldrich Fine Chemicals as Product No. 18,152-8, having a weight average molecular weight (Mw) of about 337 kilodaltons. Stents were either used as received (i.e., uncoated metal), were pre-treated with a silane/Parylene™ primer using the primer procedure described in the Sample Preparation Procedure, were not pre-treated with primer but were given a subsequent PBMA topcoat using the spraying process described in the Sample Preparation Procedure, or were given both a silane/Parylene™ pre-treatment primer and subsequent PBMA topcoat.

After preparing the coated stents and allowing all solvents to dry at ambient conditions, the stents were examined with an optical microscope under both “bright field” and “dark field” conditions. All coatings were optically transparent (i.e., clear, showing no cloudiness). Raman microscopy taken of the coated stents of PEMA as first polymer, applied to bare metal stent, indicated a high degree of homogeneity of mixing of drug and polymers.

The coated stents were crimped down on balloons and were expanded following the Durability Test Procedure, which showed that, overall, all the coatings remained intact (i.e., the coating did not peel off or flake off, etc.), with only a few localized sites where coating delaminated from the metal stent. When primer coatings were used, essentially no delamination was evident and cracks were all smaller than about 10 microns in width. Almost all stents had some degree of cracking of the coating around bends in the struts, as well as some mechanical damage caused by handling or balloon expansion. Adding a PBMA topcoat did not adversely affect the mechanical integrity of the coating on the stent after crimping and expansion, as might be expected with an overall thicker stent coating.

Based on both the drug-eluting test results and mechanical test results, coatings containing bioactive agents incorporated into blends of PBMA with PEMA as the first polymer are viable candidates for commercial applications in drug-eluting stents and are expected to be particularly effective in minimizing the onset of restenosis after stent implantation.

### Example 8 and Comparative Example C1

### Stress-Strain Measurements for First and/or Additional Polymers

Tensile properties of various first polymers and additional polymers of this invention were tested and average tensile modulus calculated using the Stress-Strain Measurement Test Procedure. The first and/or additional polymers evaluated were poly(ethylene-co-methyl acrylate) (“PEMA”, same as used in Example 5), poly(ethylene-co-butyl acrylate) (“PEBA”, containing 35% (wt) butyl acrylate, available from Focus Chemical Corp., Portsmouth, N.H.), polybutadiene (“PBD”, available from Scientific Polymers Products, Inc., Ontario, N.Y., as Catalog # 688; CAS #31567-90-5; 7% cis 1,4; 93% vinyl 1,2; M_{w} approx. 100 kilodaltons) and poly(ethylene-co-vinyl acetate) (“PEVA”, available as Product No. 34,691-8 from Sigma-Aldrich Fine Chemicals). PEVA was run as a comparative example.

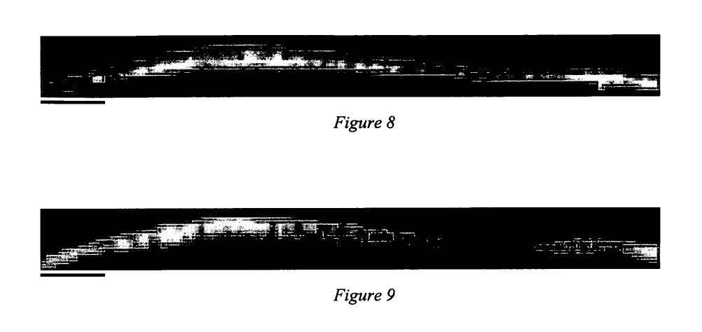

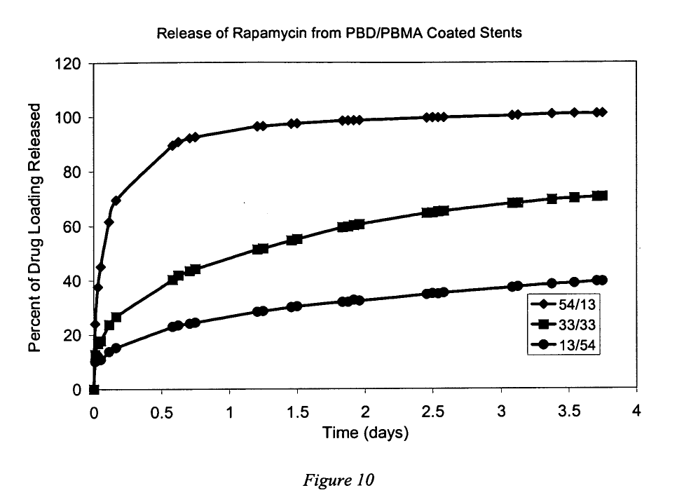

Comparison of FIGS. 8 and 9 indicates that the bioactive agent is uniformly distributed within the entire coating, since the intensity of the Raman signal of the agent varies only subtly from one region of the coating to another. Similar results are seen with other compositions of the present invention.

### Example 10

### Release of Rapamycin from Poly(butadiene) and Poly(butyl methacrylate)

Three solutions were prepared for coating the stents. The solutions included mixtures of poly(1,2-butadiene) (“PBD”, available from Scientific Polymers Products, Inc., Ontario, N.Y., as Catalog # 688; CAS #31567-90-5; 7% cis 1,4; 93% vinyl 1,2; Mw approx. 100 kilodaltons), poly(butyl methacrylate) (“PBMA”, available from Sigma-Aldrich Fine Chemicals as Product No. 18,152-8, having a weight average molecular weight (Mw) of about 337 kilodaltons), and rapamycin (“RAPA”, available from LC Laboratories, Woburn, Mass.) dissolved in THF to form a homogeneous solution. The stents were not given a primer pre-treatment.

The solutions were prepared to include the following ingredients at the stated weights per milliliter of THF:
1) 16 mg/ml PBD/4 mg/ml PBMA/10 mg/ml RAPA
2) 10 mg/ml PBD/10 mg/ml PBMA/10 mg/ml RAPA
3) 4 mg/ml PBD/16 mg/ml PBMA/10 mg/ml RAPA

Using the Sample Preparation Procedure, two stents were spray coated using each solution. After solvent removal via ambient evaporation, the drug elution for each coated stent was monitored using the Rapamycin Release Assay Procedure.

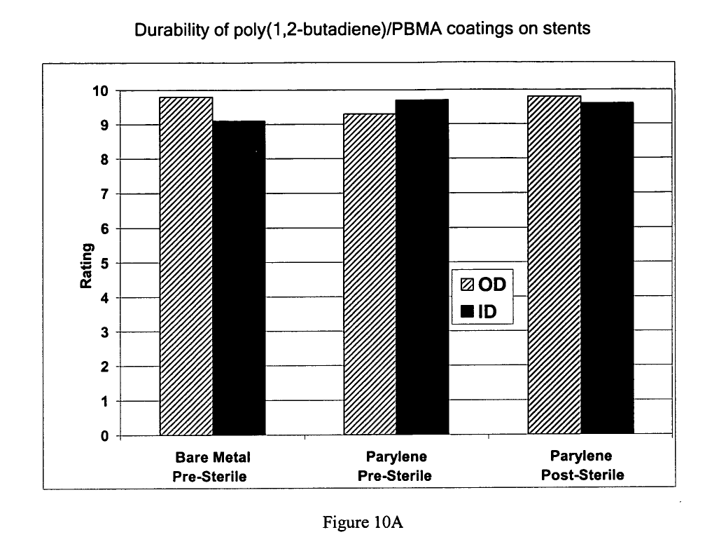

Results, provided in FIG. 10, demonstrate the ability to control the elution rate of rapamycin, a pharmaceutical agent, from a coated stent surface by varying the relative concentrations of PBD and PBMA in the polymer mixture as described herein. The lines in FIG. 10 and similar figures are expressed in terms of percent by weight of the first and second polymers, respectively, in the coated compositions. This can be compared to the amounts provided above, which are stated in terms of “mg/ml” of the respective polymers in the coating compositions themselves, which are applied to the stents. Hence “54/13” corresponds to the coated compositions that results from the use of the first coating composition above, which upon removal of the solvent provides a coated composition having 54% PBD and 13% PBMA respectively, by weight. Alternatively, solutions such as the second solution above, e.g., which includes equal amounts (by weight) of the ingredients, will alternatively be referred to herein as “33/33/33”, representing the weight ratio of ingredients to each other.

Additionally, the durability for PBD/PBMA coatings was also analyzed. Stents were coated with PBD and PBMA in a procedure as described above but without any bioactive agent. The stents were then tested according to the method described in the Durability Test Procedure section. The results are displayed in FIG. 10A. The PBD/PBMA coatings showed very little damage in the form of some small cracks that did not appear to reach the stent surface. These coatings were applied to bare metal stents before ethylene oxide sterilization (“sterilization”), Parylene™ coated stents before sterilization, and Parylene™ coated stents after sterilization. These were labeled in FIG. 10A “Bare Metal Pre-Sterile,” “Parylene Pre-Sterile,” and “Parylene Post-Sterile,” respectively. Parylene™ treatments and sterilization had little effect on the exceptional durability of the PBD/PBMA coatings.

### Example 11

### Release of Rapamycin from Poly(butadiene-co-acrylonitrile) and Poly(butyl methacrylate)

Three solutions were prepared for coating the stents. The solutions included mixtures of poly(butadiene-co-acrylonitrile) (“PBDA,” available from Scientific Polymer Products, Inc., Catalog #533, contains 41% (wt) acrylonitrile), “PBMA” and “RAPA” (“PBMA” and “RAPA” were obtained and used as described in Example 1) dissolved in THF to form a homogeneous solution. The stents were not given a primer pre-treatment.

The solutions were prepared to include the following ingredients at the stated weights per milliliter of THF:
1) 16 mg/ml PBDA/4 mg/ml PBMA/10 mg/ml RAPA
2) 10 mg/ml PBDA/10 mg/ml PBMA/10 mg/ml RAPA
3) 4 mg/ml PBDA/16 mg/ml PBMA/10 mg/ml RAPA

Using the Sample Preparation Procedure, two stents were spray coated using each solution. After solvent removal via ambient evaporation, the drug elution for each coated stent was monitored using the Rapamycin Release Assay Procedure.

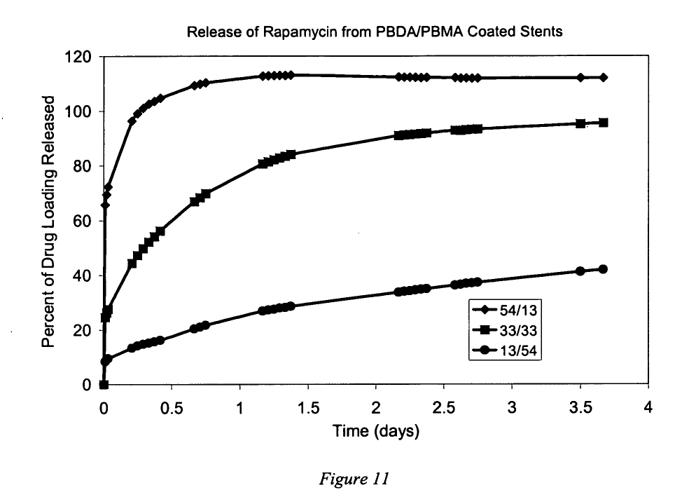

Results, provided in FIG. 11, demonstrate the ability to control the elution rate of rapamycin, a pharmaceutical agent, from a coated stent surface by varying the relative concentrations of PBDA and PBMA in the polymer mixture as described herein.

### Example 12

### Release of Dexamethasone from Poly(butadiene) and Poly(butyl methacrylate)

Three solutions were prepared for coating the stents. All three solutions included mixtures of poly(1,2-butadiene) “PBD”, poly(butyl methyl acrylate) (“PBMA”), and dexamethasone (“DEXA”) dissolved in THF to form a homogeneous solution. The stents were not given a primer pre-treatment.

The solutions were prepared to include the following ingredients at the stated weights per milliliter of THF:
1) 20 mg/ml PBD/0 mg/ml PBMA/10 mg/ml DEXA
2) 10 mg/ml PBD/10 mg/ml PBMA/10 mg/ml DEXA
3) 0 mg/ml PBD/20 mg/ml PBMA/10 mg/ml DEXA

Using the Sample Preparation Procedure, two stents were spray coated using each solution. After solvent removal via ambient evaporation, the drug elution for each coated stent was monitored using the Dexamethasone Release Assay Procedure.

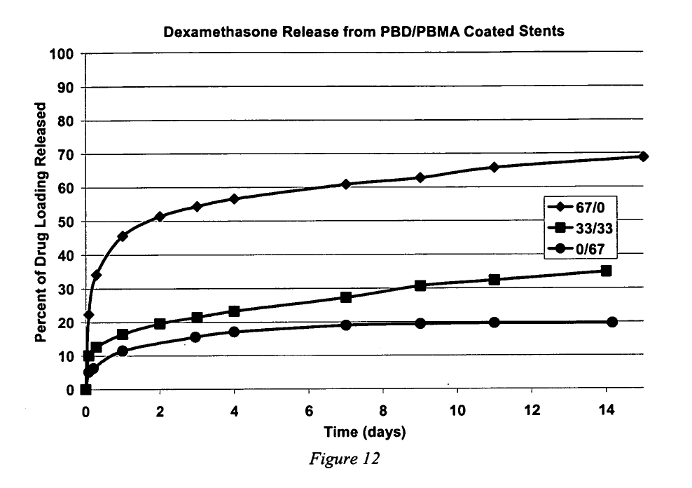

Results, provided in FIG. 12, demonstrate the ability to control the elution rate of dexamethasone, a pharmaceutical agent, from a stent surface by varying the relative concentrations of PBD and PBMA in the polymer mixture.

### Example 13

### Surface Characterization of Coated Stents after Crimping and Expansion

Using the Sample Preparation Procedure, stents were sprayed with a coating of second polymer/poly(butyl methacrylate)(“PBMA”)/rapamycin(“RAPA”), mixed at a weight ratio of 33/33/33 at 10 mg/ml each of THF. The first polymer was polybutadiene (“PBD”, available from Scientific Polymers Products, Inc., Ontario, N.Y., as Catalog # 688; CAS #31567-90-5; 7% cis 1,4; 93% vinyl 1,2; M_{w} approx. 100 kilodaltons), and a polymer from the additional polymer class was poly(ethylene-co-methyl acrylate) (“PEMA”, available from Focus Chemical Corp. Portsmouth, N.H., containing 28% (wt) methyl acrylate). The second polymer used was PBMA from Sigma-Aldrich Fine Chemicals as Product No. 18,152-8, having a weight average molecular weight (Mw) of about 337 kilodaltons. Stents were either used as received (i.e., uncoated metal), were pre-treated with a silane/Parylene™ primer using the primer procedure described in the Sample Preparation Procedure, were not pre-treated with primer but were given a subsequent PBMA topcoat using the spraying process described in the Sample Preparation Procedure, or were given both a silane/Parylene™ pre-treatment primer and subsequent PBMA topcoat.

After preparing the coated stents and allowing all solvents to dry at ambient conditions, the stents were examined with an optical microscope under both “bright field” and “dark field” conditions. All coatings were optically transparent (i.e., clear, showing no cloudiness). Raman microscopy taken of the coated stents of (PEMA as the additional polymer, applied to bare metal stent) and (PBD as the first polymer, applied to bare metal stent) indicated a high degree of homogeneity of mixing of drug and polymers.

The coated stents were crimped down on balloons and were expanded following the Durability Test Procedure, which showed that, overall, all the coatings remained intact (i.e., the coating did not peel off or flake off, etc.), with only a few localized sites where coating delaminated from the metal stent. When primer coatings were used, essentially no delamination was evident and cracks were all smaller than about 10 microns in width. Almost all stents had some degree of cracking of the coating around bends in the struts, as well as some mechanical damage caused by handling or balloon expansion. Adding a PBMA topcoat did not adversely affect the mechanical integrity of the coating on the stent after crimping and expansion, as might be expected with an overall thicker stent coating.

Based on both the drug-eluting test results and mechanical test results, coatings containing bioactive agents incorporated into blends of PBMA with either PEMA or PBD as the other polymer are viable candidates for commercial applications in drug-eluting stents and are expected to be particularly effective in minimizing the onset of restenosis after stent implantation.

### Example 14

### Scanning Electron Microscopy

Scanning Electron Microscopy can be used to observe coating quality and uniformity on stents at any suitable point in their manufacture or use. Crimped and expanded stents were examined for coating failures in fine microscopic detail using a scanning electron microscope (SEM) at magnifications varying from 150× to 5000×.

Various coating defects tend to affect the manufacture and use of most polymer coated stents in commercial use today, including the appearance of cracks or tears within the coating, smearing or displacement of the coating, as well as potentially even delamination of the coating in whole or in part. Such defects can occur upon formation of the coating itself, or more commonly, in the course of its further fabrication, including crimping the stent upon an inflatable balloon, or in surgical use, which would include manipulating the stent and expanding the balloon to position the stent in vivo.

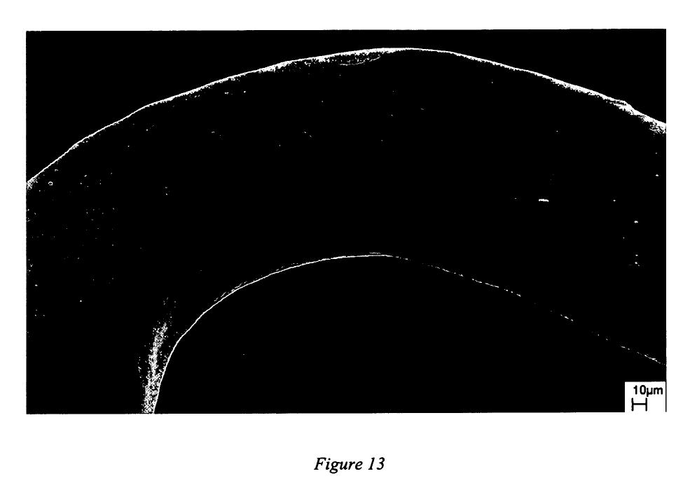

FIG. 13 shows a scanning electron microscope image from a LEO Supra-35 VP at 250× of a 33/33/33 PBD/PBMA/rapamycin coating on a stent after conventional crimping and balloon expansion procedures. The image shows that the coated composition maintains integrity after expansion, showing no evidence of delamination or cracks.

When observed by SEM, many other compositions tended to show cracks, however, typically of a type and number that are certainly on par with those in commercial use today, and would tend to be well within acceptable range, particularly considering that neither the coating compositions, or manner of applying particular compositions, have yet been optimized for any particular combination of surface, polymers, bioactive agent. The cracks were typically a few microns in width, with thin strands of polymer stretching between the edges of the crack. Overall, however, the coatings looked smooth, uniform, and in good condition.

Almost all the stents had some degree of cracking of the coating around bends in the struts, as well as some mechanical damage caused by handling or balloon expansion. Most surprisingly, polybutadiene-containing coatings exhibited less cracking and in one case no cracks, and when cracks occurred, they were typically smaller in size in comparison with the cracks found in PEMA or PEVA-containing coatings. For comparison, cracks which opened up and delaminated from the metal stent surface were found in coatings containing PEMA and PEVA in the absence of a Parylene™ primer coating. Polybutadiene-containing coatings without Parylene™ primer, as well as comparative PEMA (or comparative PEVA)-containing coatings with Parylene™ primer, showed cracks which tended to not result in delamination.

### Example 15

### Release of Rapamycin from Poly(butadiene) and Poly(butyl methacrylate) Provided with a Topcoat

Several solutions were prepared for coating non-sterile, non-deployed, self-expanding nitinol coronary stents having a primer layer. The solutions included mixtures of poly(1,2-butadiene) (“PBD”, available from Scientific Polymers Products, Inc., Ontario, N.Y.), poly(butyl methacrylate) (“PBMA”, available from Sigma-Aldrich Fine Chemicals), and rapamycin (“RAPA”, available from LC Laboratories, Woburn, Mass.) dissolved to form a homogeneous solution. In addition, a topcoat of PBMA was also prepared and applied to the coating composition on some of the stents, and the elution rate profiles into a 2% SLS buffer on a Sotax USP IV Apparatus were determined.

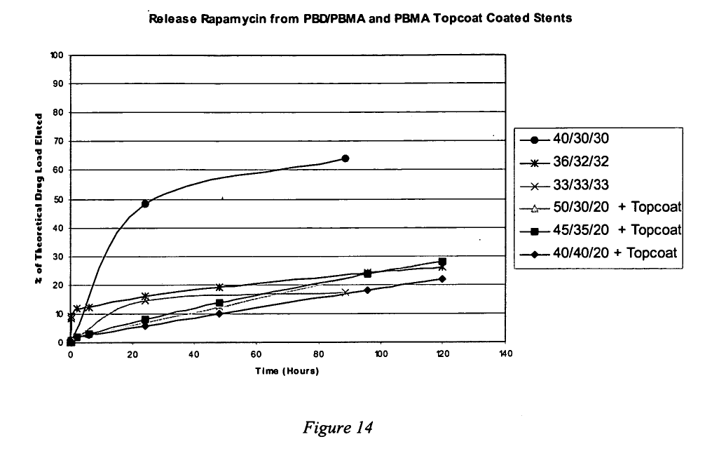

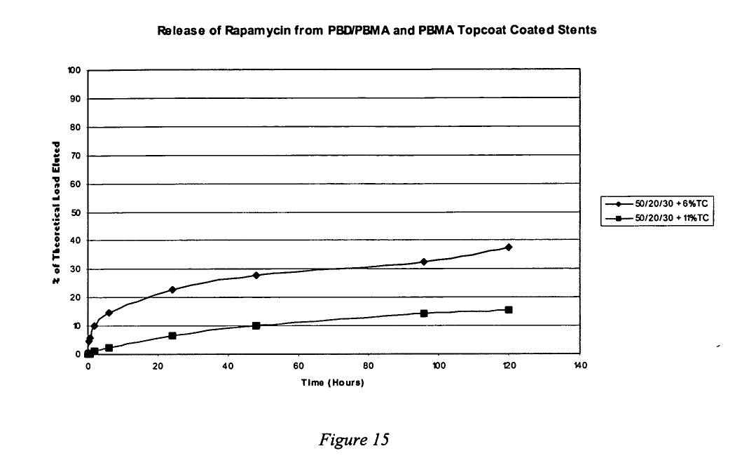

Results, provided in FIG. 14, illustrates several elution rates of rapamycin, a pharmaceutical agent, from a coated stent surface by varying the relative concentrations of rapamycin, PBD, and PBMA with and without utilizing a topcoat. Further, FIG. 15 demonstrates the ability to control the elution rate of a bioactive agent by varying the amount of topcoat provided relative to the coating composition.

The lines in FIG. 14 and FIG. 15 are expressed in terms of percent by weight of the Rapamycin, PBD, and PBMA, respectively, in the coated compositions. Hence “40/30/30” corresponds to the coated compositions that results from the use of 40% Rapamycin, 30% PBD, and 30% PBMA, respectively, by weight. In FIG. 15, the weight of the topcoat relative to the weight of the coating composition is shown. For example, 6% topcoat corresponds to an amount of topcoat totaling 6% by weight of the coating composition weight.

### Example 16

### Release of Sirolimus from Poly(Butadiene) and Poly(butyl methacrylate) with Poly(butyl methacrylate) and Sirolimus Topcoats

Stainless steel BX velocity stents manufactured by Cordis Corporation, Miami Lakes, Fla. were used in the following examples. The stents were Parylene treated and weighted before coating.

Bioactive agent/polymer solutions were prepared at a range of concentrations in an appropriate solvent, in the manner described herein. The coating solutions were applied to respective stents by spraying procedures using an ultrasonic sprayer as described in U.S. Published Application 2004/0062875 (Chappa et al.); and in U.S. application Ser. No. 11/102,465, filed Apr. 8, 2005 and entitled “Medical Devices and Methods for Producing Same.” After spraying application of the bioactive agent/polymer solution, the solvent was allowed to evaporate. The coated stents were weighed to determine the mass of coating and consequently the mass of polymer and bioactive agent. The coating thickness can be measured using any suitable means, e.g. optical interferometry.

The Bioactive Agent Release Assay, as described herein, was used to determine the extent and rate of drug release in vitro conditions. A Sotax dissolution system (Sotax Corporation, Horsham, Pa.) was utilized. The system used a 2 wt % surfactant/water solution as elution media. The coated stents were placed in the sample baskets, and the drug elution monitored by UV spectrometry over the course of several days. The elution media was held at 37° C. After the elution measurement, the stents were removed, rinsed, dried, and weighed to compare measured drug elution to mass loss.

One basecoat solution was prepared for coating the stents. This solution included mixtures of “PBD” poly(butadiene), “PBMA” poly(butyl methacrylate), and sirolimus dissolved in tetrahydrofuran (THF). The basecoat solution contained 6 mg/ml PBD, 6 mg/ml PBMA, and 6 mg/ml Sirolimus for a total “solids” concentration of 18 mg/ml. Stents were coated with approximately 435 micrograms of total coating. The basecoat was allowed to dry before the topcoats were applied.

The following THF solutions were used for the topcoats:
1) 18 mg/ml PBMA and 2 mg/ml Sirolimus
2) 12 mg/ml PBMA and 8 mg/ml Sirolimus
3) 20 mg/ml PBMA

Average topcoat weights were 121 micrograms, and two stents were spray-coated using each solution.

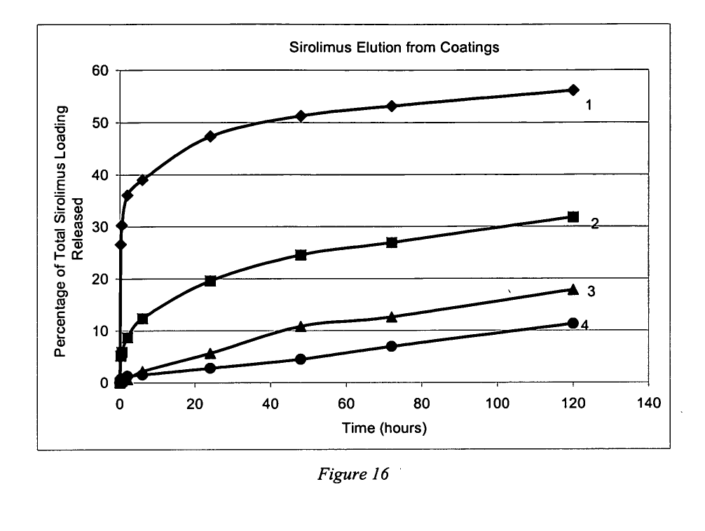

After the solvent was removed by evaporation, drug elution was tested via the bioactive agent release assay described above. The results are provided in FIG. 16 where curve 1 is basecoat only, curve 2 is topcoat applied using coating solution 1, curve 3 is topcoat applied using coating solution 2, and curve 4 is topcoat applied using coating solution 3. These curves demonstrated the ability to control the elution rate of a bioactive agent from a medical device surface by varying the amount of bioactive agent in the topcoat.

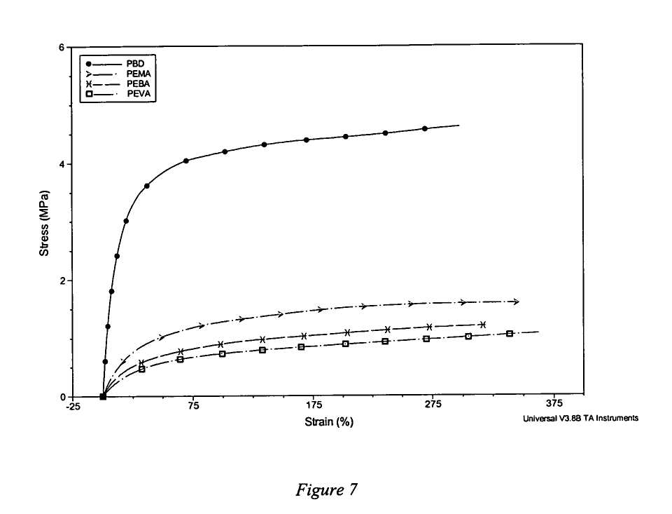

Stress-strain curves are shown in FIG. 7. The calculated average tensile modulus for each of the tested polymers is shown in Table 1.

TABLE 1

| Example | Polymer | Average Tensile Modulus, MPa (SD) |
|---|---|---|
| 8a | PEMA | 5.54 (0.49) |
| 8b | PEBA | 3.66 (0.67) |
| 8c | PBD | 34.87 (4.83) |
| C1 | PEVA | 2.17 (0.46 |

The data from Table 1 show that, when compared to PEVA, each of the first polymers showed a higher average tensile modulus. The average tensile modulus for the PBD was significantly higher than that for any of the other polymers.

### Example 9

### Raman Microscopy

Raman measurements were made with a WITec CRM200 scanning confocal Raman microscope. The Raman microscope can optically dissect a layer of coating on a stent, looking into the coating and imaging the distribution of the coating composition ingredients within the thin coating. Since no Raman signal is obtained from air and steel materials, the air above the coating surface is black as is the steel substrate upon which the coating is deposited.

FIG. 8 shows a 100 micron wide and 10 micron deep image (including a 10 micron bar in the lower left-hand corner for scale) taken by measuring the Raman intensity at 2900 cm⁻¹ for a stent with a 33/33/33 PEMA/PBMA/rapamycin coating. Since each of the composition ingredients, including first and second polymers as well as bioactive agent, contribute signal at this wavenumber, the image obtained is one of the entire coating. FIG. 9 shows Raman intensity at 1630 cm⁻¹ for the same region of stent coating shown in FIG. 8. When measuring the Raman intensity at 1630 cm⁻¹, only the intensity of the bioactive agent signal is measured (the first and second polymers do not emit at this wavenumber), and so an image of the distribution of the bioactive agent within the coating is obtained (FIG. 9).

### Example 17

### Pretreating the Surface of Medical Device by Roughening

Eye coils were roughened by blasting 50 μm silica particles at the surface of the coils under high pressure and velocity. The roughness of the coil surfaces, particularly the peak to valley distance, was measured with the VSI (Vertical Scanning Interferometry) mode of an Optical Interferometer.

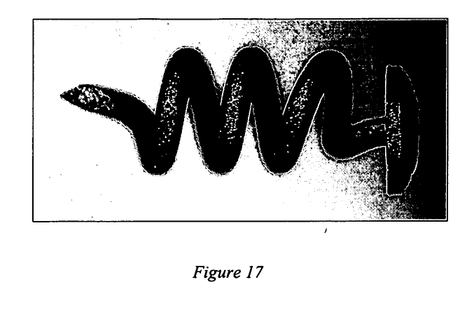

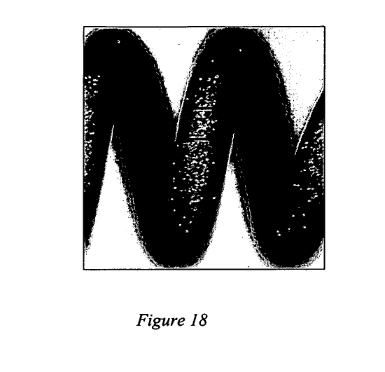

Roughness tests were taken over areas approximately 155 μm×120 μm on the top of each turn in the coils, as shown in FIGS. 17 and 18. Three roughness tests were taken on one side of the coil, then the coil was rotated 180° and three more tests were taken on the other side of the coil.

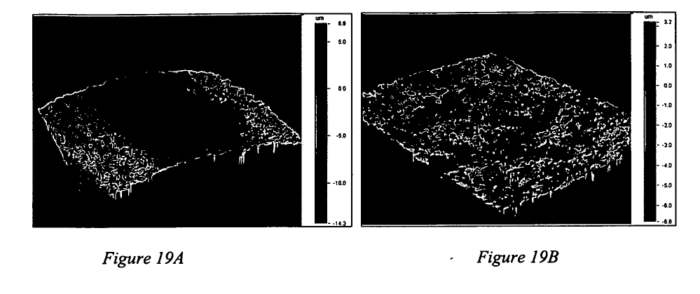

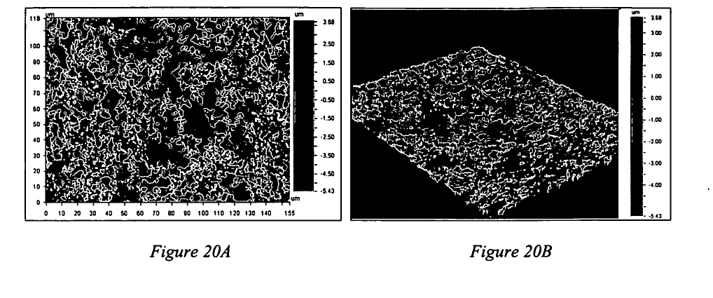

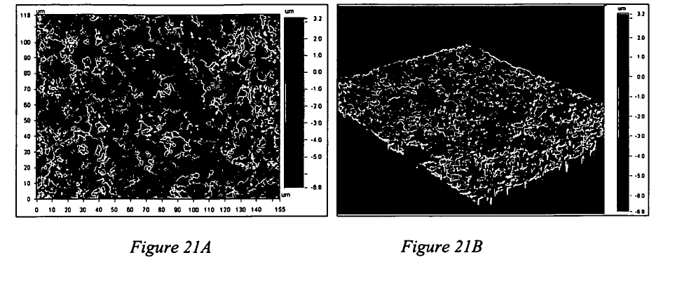

The VSI mode of the optical interferometer was used to look at the surface topography of uncoated eye coils over an area approximately 155 um×120 μm. Three separate areas were measured on each coil on two sides of each coil, to get an average for each. Each measurement comprised of a 30 μm scan to acquire the raw data, after which Rₐ, Rₜ, and R_{z} roughness parameters were calculated. Rₐ, the roughness average, is the arithmetic mean of the absolute values of the surface departures from the mean plane. Rₜ, the maximum height (peak to valley distance), is the vertical distance between the highest and lowest points over the entire dataset (highest and lowest single pixels), R_{z}, the average maximum height (average peak to valley distance), is the average of the difference of the ten highest and ten lowest points in the dataset (10 highest and 10 lowest pixels at least 4.6 μm apart from each other laterally). The R_{z} value measures the average peak to valley distance from multiple locations to prevent a misrepresentation of the data caused by single data pixels that are random noise, or uncommon surface features like scratches or pits. As shown below in FIGS. 19A and B, tilt and curvature of the surface were removed in order to compare the relative surface finish of each coil. Table 2 shows the roughness statistics for coil 1, and Table 3 shows the roughness statistics for coil 2. FIG. 20A shows a surface plot of test A-2 of coil 1, and FIG. 20B shows a 3D representation of FIG. 20A. FIG. 21A shows a surface plot of test A-2 of coil 2, and FIG. 21B shows a 3D representation of FIG. 21A.

TABLE 1
Coil #1 Roughness Statistics

| Test Position | Rₐ (nm) | Rₜ (μm) | R_{z} (μm) |
|---|---|---|---|
| A-1 | 625.03 | 8.51 | 6.78 |
| A-2 | 756.07 | 9.11 | 7.84 |
| A-3 | 686.93 | 13.92 | 10.75 |
| B-1 | 795.54 | 9.24 | 8.29 |
| B-2 | 782.50 | 15.27 | 11.78 |
| B-3 | 778.56 | 10.46 | 8.95 |
| Avg. +/− St. Dev. | 737.44 ± 67.28 | 11.09 ± 2.82 | 9.07 ± 1.87 |

TABLE 3
Coil #2 Roughness Statistics

| Test Position | Rₐ (nm) | Rₜ (μm) | R_{z} (μm) |
|---|---|---|---|
| A-1 | 790.22 | 10.88 | 8.34 |
| A-2 | 626.39 | 10.01 | 7.35 |
| A-3 | 1170.03 | 11.41 | 10.11 |
| B-1 | 628.17 | 10.77 | 7.87 |
| B-2 | 727.82 | 13.00 | 8.98 |
| B-3 | 863.89 | 10.91 | 8.94 |
| Avg. +/− St. Dev. | 801.08 ± 202.96 | 11.17 ± 1.01 | 8.60 ± 0.97 |

Other embodiments of this invention will be apparent to those skilled in the art upon consideration of this specification or from practice of the invention disclosed herein. Various omissions, modifications, and changes to the principles and embodiments described herein may be made by one skilled in the art without departing from the true scope and spirit of the invention which is indicated by the following claims. All patents, patent documents, and publications cited herein are hereby incorporated by reference as if individually incorporated.

What is claimed is:

1. A composition for coating the surface of a medical device with a bioactive agent in a manner that permits the coated surface to release the bioactive agent over time when implanted in vivo, the composition comprising a bioactive agent in combination with a plurality of polymers, including a first polymer component and a second polymer component, and further comprising a topcoat in apposition to the composition, the topcoat including the polymer of the second polymer component in the composition and a topcoat bioactive agent.

2. The composition of claim 1, wherein the bioactive agent is distinguishable from the topcoat bioactive agent.

3. The composition of claim 1, wherein the first polymer component comprises at least one polymer selected from the group consisting of ethylene copolymers with other alkylenes, polybutenes, aromatic group-containing copolymers, epichlorohydrin-containing polymers, poly(alkylene-co-alkyl(meth)acrylates), and diolefin-derived, non-aromatic polymers and copolymers.

4. The composition of claim 1, wherein the second polymer component comprises a polymer selected from the group consisting of poly(alkyl(meth)acrylates) and poly(aromatic(meth)acrylates).

5. The composition of claim 1, further including the medical device, the medical device having a roughened surface to increase the adhesion of the coating composition to the medical device and/or alter the elution rate of the bioactive agent.

6. The composition of claim 1, further including an additional polymer.

7. A composition for coating the surface of a medical device with a bioactive agent in a manner that permits the coated surface to release the bioactive agent over time when implanted in vivo, the composition comprising a bioactive agent in combination with a plurality of polymers, including a first polymer component comprising at least one polymer selected from the group consisting of ethylene copolymers with other alkylenes, polybutenes, aromatic group-containing copolymers, epichlorohydrin-containing polymers, poly(alkylene-co-alkyl(meth)acrylates), and diolefin-derived, non-aromatic polymers and copolymers and a second polymer component comprising a polymer selected from the group consisting of poly(alkyl(meth)acrylates) and poly(aromatic(meth)acrylates), and further comprising a topcoat in apposition to the composition, the topcoat including the polymer of the second polymer component in the composition and a topcoat bioactive agent.

8. The composition of claim 7, wherein the bioactive agent is distinguishable from the topcoat bioactive agent.

9. The composition of claim 7, further including the medical device, the medical device having a roughened surface to increase the adhesion of the coating composition to the medical device and/or alter the elution rate of the bioactive agent.

10. A composition for coating the surface of a medical device with a bioactive agent in a manner that permits the coated surface to release the bioactive agent over time when implanted in vivo, the composition comprising a bioactive agent in combination with a plurality of polymers, including a first polymer component and a second polymer component, and further comprising a topcoat in apposition to the composition, the topcoat including a topcoat bioactive agent, the topcoat reducing the elution rate of a bioactive agent from a medical device surface.

11. The composition of claim 10, wherein the topcoat is relatively thin compared to the composition.

12. The composition of claim 10, wherein the topcoat reduces bioactive agent elution rates.

13. The composition of claim 10, wherein the topcoat weighs less than about five percent of the composition and reduces elution rates by more than about fifty percent for at least about twenty hours compared to compositions without topcoats.

14. The composition of claim 10, wherein the first polymer component comprises at least one polymer selected from the group consisting of ethylene copolymers with other alkylenes, polybutenes, aromatic group-containing copolymers, epichlorohydrin-containing polymers, poly(alkylene-co-alkyl(meth)acrylates), and diolefin-derived, non-aromatic polymers and copolymers

15. The composition of claim 10, wherein the second polymer component comprises a polymer selected from the group consisting of poly(alkyl(meth)acrylates) and poly(aromatic(meth)acrylates).

16. The composition of claim 10, further including the medical device, the medical device having a roughened surface to increase the adhesion of the coating composition to the medical device and/or alter the elution rate of the bioactive agent.

17. A composition for coating the surface of a medical device with a bioactive agent in a manner that permits the coated surface to release the bioactive agent over time when implanted in vivo, the composition comprising a bioactive agent in combination with a plurality of polymers, including a hydrophobic first polymer component and a hydrophobic second polymer component, and further comprising a hydrophilic topcoat.

18. The composition of claim 17, the hydrophilic topcoat comprising an agent selected from the group consisting of polyacrylamide(36%)co-methacrylic acid(MA)-(10%)co-methoxy PEG1000MA-(4%)co-BBA-APMA, photoheparin, and a photoderivatized coating agent.

19. The composition of claim 17, wherein the hydrophobic first polymer component comprises at least one polymer selected from the group consisting of ethylene copolymers with other alkylenes, polybutenes, aromatic group-containing copolymers, epichlorohydrin-containing polymers, poly(alkylene-co-alkyl(meth)acrylates), and diolefin-derived, non-aromatic polymers and copolymers.

20. The composition of claim 17, wherein the hydrophobic second polymer component comprises a polymer selected from the group consisting of poly(alkyl(meth)acrylates) and poly(aromatic(meth)acrylates).

21. A method of controlling the elution rate of one or more bioactive agents from a coated surface of a medical device comprising:
administering a bioactive agent coating including a composition comprising a bioactive agent in combination with a plurality of polymers, including a first polymer component and a second polymer component to a surface; and
administering a topcoat over the bioactive agent coating, the topcoat including one or more bioactive agents.

22. The method of controlling the elution rate of one or more bioactive agents from a coated surface of a medical device of claim 21 wherein the topcoat includes a one or more materials selected from the group consisting of a first polymer component, a second polymer component, parylene, photochemical materials, thermochemical materials and hydrophilic materials.

23. The method of controlling the elution rate of one or more bioactive agents from a coated surface of a medical device of claim 22 wherein the topcoat includes a second polymer component selected from one or more polyalkyl(meth)acrylates.

24. The method of controlling the elution rate of one or more bioactive agents from a coated surface of a medical device of claim 22 wherein the topcoat includes one or more photochemical or thermochemical materials selected from the group consisting of photo-heparin and photo-collagen.

25. The method of controlling the elution rate of one or more bioactive agents from a coated surface of a medical device of claim 22 wherein the topcoat includes a hydrophilic material selected from the group consisting of polyacrylamide(36%)co-methacrylic acid(MA)-(10%)co-methoxy PEG1000MA-(4%)co-BBA-APMA and photoheparin.

26. A combination including a medical device and a composition for coating the surface of the medical device with a bioactive agent in a manner that permits the coated surface to release the bioactive agent over time when implanted in vivo, the composition comprising a bioactive agent in combination with a plurality of polymers, including a first polymer component and a second polymer component, the medical device having a roughened surface to increase the adhesion of the coating composition to the medical device and/or alter the elution rate of the bioactive agent.

27. The combination of claim 26, wherein the first polymer component comprises at least one polymer selected from the group consisting of ethylene copolymers with other alkylenes, polybutenes, aromatic group-containing copolymers, epichlorohydrin-containing polymers, poly(alkylene-co-alkyl(meth)acrylates), and diolefin-derived, non-aromatic polymers and copolymers.

28. The combination of claim 26, wherein the second polymer component comprises a polymer selected from the group consisting of poly(alkyl(meth)acrylates) and poly(aromatic(meth)acrylates).

29. The combination of claim 26, wherein the extent of roughening ranges from about 2 μm to about 20 μm.

30. The combination of claim 26, wherein the medical device comprises an ocular coil.
